(19) United States
(12) Patent Application Publication
(10) Pub. No.: US 2014/0322314 A1
(43) Pub. Date: Oct. 30, 2014

(54) Omega-3 Fatty Acid Formulations for Use as Pharmaceutical Treatment

(71) Applicant: Matinas BioPharma, Inc., Bedminster, NJ (US)

(72) Inventors: Abdel Aziz Fawzy, Boynton Beach, FL (US); George Bobotas, Tarpon Springs, FL (US)

(73) Assignee: Matinas BioPharma, Inc., Bedminster, NJ (US)

(21) Appl. No.: 14/264,990

(22) Filed: Apr. 29, 2014

Related U.S. Application Data
(60) Provisional application No. 61/817,296, filed on Apr. 29, 2013.
(60) Provisional application No. 61/817,310, filed on Apr. 29, 2013.

(51) Int. Cl. A61K 47/18; A61K 47/12; A61K 47/10; A61K 31/202; A61K 47/14

(52) U.S. Cl. CPC A61K 47/186; A61K 31/202; A61K 47/14; A61K 47/10; A61K 47/12
USPC 424/456; 514/560

(57) ABSTRACT

Pharmaceutical formulations comprising at least 30% omega-3-fatty acids, salts or derivatives thereof, and one or more additional components selected from the group consisting of: absorption enhancers and adjuvants These formulations have improved pharmacological features, and can be used for the treatment or prophylaxis of dyslipidemic, cardiovascular, CNS, inflammatory, and other diseases/conditions or risk factors therefore.

### RELATED APPLICATION

This application claims the benefit of U.S. Provisional Patent Application No. 61/817,296, filed Apr. 29, 2013, and U.S. Provisional Patent Application No. 61/817,310, filed Apr. 29, 2013, the contents of which are incorporated herein by reference.

### FIELD OF INVENTION

The present invention relates to compositions comprising omega-3 fatty acids, salts or derivatives thereof, and one or more additional components selected from the group consisting of: absorption enhancers and adjuvants.

The present invention relates to a method utilizing compositions comprising omega-3 fatty acids for the treatment of patients by administering an effective amount of a composition of the present invention to a subject prone to or afflicted with such disease, and wherein the disease is an amenable disease selected from the group consisting of: hypertriglyceridemia; hypercholesterolemia; mixed dyslipidemia; coronary heart disease (CHD); vascular disease; atherosclerotic disease and related conditions; heart failure; cardiac arrhythmias; ischemic dementia; hypertension; coagulation related disorders; nephropathy; kidney or urinary tract disease; retinopathy; cognitive and other CNS disorders; autoimmune diseases; inflammatory diseases; asthma or other respiratory disease; dermatological disease; metabolic syndrome; diabetes or other form of metabolic disease; liver disease; disease of the gastrointestinal tract; disease of the male or female reproductive system or related secondary sexual organs; a cancer; an infection caused by a virus, bacterium, fungus, protozoa or other organism; and the treatment and/or prevention and/or reduction of cardiac events and/or cardiovascular events and/or vascular events and/or symptoms. The present invention also relates to treatment of such conditions in with concomitant treatments regimes or combination products with other active pharmaceutical ingredients.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-cholesterol, and apolipoprotein B (a membrane complex for LDL-cholesterol and VLDL-cholesterol, as well as IDL-cholesterol in rare individuals suffering from a disorder resulting in significant IDL-cholesterol levels) promote human atherosclerosis; these elevated levels are often referred to as hypercholesterolemia. Decreased levels of HDL-cholesterol and its transport complex, apolipoprotein A, as well as elevated levels of apolipoprotein C-III and serum triglycerides (TG) are also associated with the development of atherosclerosis. Further, cardiovascular morbidity and mortality in humans can vary directly with the level of total-C, LDL-cholesterol and TG and inversely with the level of HDL-cholesterol. In addition, researchers have found that non-HDL-cholesterol is an important indicator of hypertriglyceridemia (elevated triglycerides), vascular disease, atherosclerotic disease and related conditions. Therefore, non-H DL-cholesterol and fasting TG reduction has also been specified as a treatment objective in NCEP ATP III. Fasting TG is commonly used as a key measure for TG in lipid management, because it minimizes the confounding factor of TG recently absorbed from meals, including the high variability of the content of meals and high variability of post-meal (post-prandial) spikes in TG. In some preferred embodiments, we refer to fasting TG levels when we refer to triglycerides or TG.

The NCEP ATPIII treatment guidelines identify HMG-CoA reductase inhibitors (“statins”) as the primary treatment option for hypercholesterolemia. In patients with TG<500 mg/dL, LDL-cholesterol is the primary treatment parameter. Many patients, however, have increased LDL-cholesterol combined with high TG and low HDL-cholesterol, a condition also known as mixed dyslipidemia. Patients with hypercholesteremia or mixed dyslipidemia often present with high blood levels of LDL-cholesterol (i.e. greater than 190 mg/dl) and TG (i.e. levels of 200 mg/dl or higher). The use of diet and single-drug therapy does not always decrease LDL-cholesterol and TG adequately enough to reach targeted values in patients with mixed dyslipidemia with or without a concomitant increase in triglycerides. In these patients, a combined therapy regimen of a statin and a second anti-dyslipidemic agent is often desired. This second agent has historically been a fibrate (i.e. gemfibrozil, bezafibrate, or fenofibrate) or extended release niacin. Over the few years, the use omega-3 fatty acid concentrates in combination with a statin has been growing rapidly due to concerns about the lack of outcome benefits with fibrates (i.e. the FIELD study) or extended release niacin (i.e. the AIM-HIGH study). In patients with isolated hypertriglyceridemia, the use of omega-3 fatty acid concentrates has also grown versus fibrates and extended release niacin.

Marine oils, also commonly referred to as fish oils, are a good source of the two main omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides (TG), increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

The table directly below lists the most common omega-3 fatty acids, including their 3-letter abbreviation code. In this application, the use of any of the 3-letter abbreviations shall refer to the omega-3 fatty acid, unless otherwise indicated (e.g. DPA or DPA 22:5 (n-3) or DPA 22:5-n3 or DPA 22:5n3 or DPA-n3, which all refer to the omega-3 isomer of docosapentaenoic acid).

| Common Name for | | |
|---|---|---|
| Omega-3 Fatty Acid | Codified | |
| (+abbreviation) | Lipid Name | Chemical Name |
| Hexadecatrienoic acid (HTA) | 16:3 (n-3) | all-cis-7,10,13- |
| | | hexadecatrienoic acid |
| α-Linolenic acid (ALA) | 18:3 (n-3) | all-cis-9,12,15- |
| | | octadecatrienoic acid |
| Stearidonic acid (SDA) | 18:4 (n-3) | all-cis-6,9,12,15- |
| | | octadecatetraenoic acid |
| Eicosatrienoic acid (ETE) | 20:3 (n-3) | all-cis-11,14,17- |
| | | eicosatrienoic acid |
| Eicosatetraenoic acid (ETA) | 20:4 (n-3) | all-cis-8,11,14,17- |
| | | eicosatetraenoic acid |
| Eicosapentaenoic acid (EPA) | 20:5 (n-3) | all-cis-5,8,11,14,17- |
| | | eicosapentaenoic acid |
| Heneicosapentaenoic acid (HPA) | 21:5 (n-3) | all-cis-6,9,12,15,18- |
| | | heneicosapentaenoic acid |
| Docosapentaenoic acid (DPA) or | 22:5 (n-3) | all-cis-7,10,13,16,19- |
| Clupanodonic acid | | docosapentaenoic acid |
| Docosahexaenoic acid (DHA) | 22:6 (n-3) | all-cis-4,7,10,13,16,19- |
| | | docosahexaenoic acid |
| Tetracosapentaenoic acid (TPA) | 24:5 (n-3) | all-cis-9,12,15,18,21- |
| | | tetracosapentaenoic acid |
| Tetracosahexaenoic acid (THA) | 24:6 (n-3) | all-cis-6,9,12,15,18,21- |
| or Nisinic acid | | tetracosahexaenoic acid |

One form of omega-3 fatty acids is a concentrate of omega-3, long chain, polyunsaturated fatty acids from fish oil containing DHA ethyl esters, EPA ethyl esters as well as ethyl esters of other omega-3 fatty acids (described in USP35 for LOVAZA®) and is sold under the trademarks OMACOR® and LOVAZA®. Such a form of omega-3 fatty acid comprises at least 90% omega-3 fatty acids of which at least 80% EPA+DHA (in a ratio of 1.2:1) and is described, for example, in U.S. Pat. Nos. 5,502,077, 5,656,667 and 5,698,594. LOVAZA® (omega-3-acid ethyl esters) is indicated for the treatment of patients with hypertriglyceridemia with TG levels of 500 mg/dL or higher.

Another form of omega-3 fatty acid concentrate is sold under the trademark EPADEL® ® for the treatment of dyslipidemia. This product is described as 98% EPA ethyl ester in Lancet (Vol. 369; Mar. 31, 2007; 1090-1098) reporting on a large outcome study with EPADEL®. EPADEL® is known to contain less than 1% of any fatty acid other than EPA.

Similar to EPADEL®, another form of omega-3 fatty acid concentrate also consists almost entirely of EPA ethyl ester and is known under its developmental stage name AMR101 or its trade name VASCEPA®. This product is described in US patent application 2010/0278879 as comprising at least 95% EPA (typically referred to as 97% or at least 96% in company releases and references) and less than 1% of any other fatty acid. AMR101 was previously under development for the treatment of Huntingdon's Disease but failed in phase III clinical development. Subsequently, AMR101 was entered in a development program for hypertriglyceridemia and mixed dyslipidemia.

Yet another concentrate of omega-3, long chain, polyunsaturated fatty acids from fish oil containing approximately 75% DHA and EPA as free fatty acids is known under its developmental stage name EPANOVA™. This product is described as comprising approximately 55% EPA and 20% DHA. EPANOVA™ was previously under development for the treatment of Crohn's Disease but failed in phase III clinical development. Subsequently, EPANOVA™ was entered in a development program for hypertriglyceridemia and mixed dyslipidemia.

Generally, the bioavailability and therapeutic effect of omega-3 fatty acid compositions is dose dependent, i.e., the higher the dose, the greater the therapeutic affect and bioavailability. However, the effect of each specific omega-3 fatty acid composition may be different, and therefore the level of therapeutic effect of one composition at a given dose cannot necessarily be inferred from the level of therapeutic effects of other omega-3 fatty acid compositions at the same or similar dose.

For instance, in the MARINE study, it was found that four 1-gram capsules of AMR101/VASCEPA® significantly reduced fasting TG in patients with very high triglycerides (TG>500 mg/dL) (March 2011, ACC poster reporting top-line results of the MARINE study), similar to four 1-gram capsules of LOVAZA® but in a less potent manner (LOVAZA® prescribing information, December 2010). In this same study, AMR101 slightly and non-significantly changed LDL-C while LOVAZA® shows a large significant increase in this same population, putting the latter at a disadvantage. Table A directly below compares these profiles.

TABLE A
Comparison of therapeutic profile of Lovaza and Vascepa in patients with very high triglycerides (>500 mg/dL)

| | LOVAZA - 4 gram/day | | Vascepa - 4 gram/day | | Vascepa - 2 gram/day | |
|---|---|---|---|---|---|---|
| | % change vs. Placebo | p-value | % change vs. Placebo | p-value | % change vs. Placebo | p-value |
| TG | −51.6 | p < 0.05 | −33.1 | p < 0.05 | −19.7 | p < 0.05 |
| Total-C | −8.0 | p < 0.05 | −16.3 | p < 0.0001 | −6.8 | p = 0.0148 |
| LDL-C | 49.3 | p < 0.05 | −2.3 | NS | 5.2 | NS |
| VLDL-C | −40.8 | p < 0.05 | −28.6 | p = 0.0002 | −15.3 | p = .038 |
| Non-HDL-C | −10.2 | p < 0.05 | −17.7 | p < 0.0001 | −8.1 | p = .0182 |
| Apo-B | NR | | −8.5 | p = 0.0019 | −2.6 | NS |
| HDL-C | 9.1 | p < 0.05 | −3.6 | NS | 1.5 | NS |

NR = Not Reported; NS = Not Significant

In another study with AMR101/VASCEPA®, the ANCHOR study, it was found that four 1-gram capsules of AMR101 significantly reduced fasting TG in patients on statin therapy with high triglycerides (TG 200-499 mg/dL), similar to four 1-gram capsules of LOVAZA® but in a less potent manner (Study in table 3, LOVAZA® prescribing information, December 2010). In this same study, AMR101 decreased LDL-C at 4 gr/day while LOVAZA® shows a significant LDL-C increase in this same population. AMR101 is also more potent than LOVAZA® in reducing non-HDL-cholesterol in this population. Table B directly below compares these profiles.

TABLE B
Therapeutic profile comparison of Lovaza and Vascepa in patients on statin with high triglycerides (TG 200-499 mg/dL)

| | LOVAZA - 4 gram/day | | Vascepa - 4 gram/day | | Vascepa - 2 gram/day | |
|---|---|---|---|---|---|---|
| | % change vs. Placebo | p-value | % change vs. Placebo | p-value | % change vs. Placebo | p-value |
| TG | −23.2 | p < 0.0001 | −21.5 | p < 0.0001 | −10.1 | p = 0.0005 |
| Total-C | −3.1 | p < 0.05 | NR | p < 0.0001 | NR | p = 0.0019 |
| LDL-C | 3.5 | p = 0.05 | −6.3 | p = 0.0067 | −3.6 | NS |
| VLDL-C | −20.3 | p < 0.05 | −24.4 | p < 0.0001 | −10.5 | p = 0.0093 |
| Non-HDL-C | −6.8 | p < 0.0001 | −13.6 | p < 0.0001 | −5.5 | p = 0.0054 |
| Apo-B | −2.3 | p < 0.05 | −9.3 | p < 0.0001 | −3.8 | p = 0.0170 |
| HDL-C | 4.6 | p < 0.05 | −4.5 | p = 0.0013 | −2.2 | NS |

NS = Not Significant

The resulting lipid profile of AMR101 versus LOVAZA® in highly similar patient populations indicates that there are significant benefits of using an almost pure EPA oil composition as opposed to an omega-3 mixture as in LOVAZA®. These benefits translate into better non-HDL- and LDL-Cholesterol reduction with the pure EPA form, where these benefits are less or, in the case of the LDL-C effect, the opposite.

The recently released results from Omthera's EVOLVE trial with EPANOVA™, in patients with very high triglycerides (TG 500 mg/dL), described a TG reduction of 31% versus baseline for the 4 gram per day dose and 26% versus baseline for the 2 gram per day dose, with 10% and 8% non-HDL reduction respectively. It appears that the TG-reducing potency of EPANOVA™ is similar to the potency of AMR101. No data were reported by Omthera on the LDL-C effect in the EVOLVE trial.

The recently released results from Omthera's ESPRIT trial with EPANOVA™, in patients with high triglycerides (TG 200-499 mg/dL) while on statin therapy, described a TG reduction of 21% versus baseline for the 4 gram per day dose and 15% versus baseline for the 2 gram per day dose, with 7% and 4% non-HDL reduction respectively. It appears that the TG-reducing potency of EPANOVA™ is similar to the potency of AMR101. No data were reported by Omthera on the LDL-C effect in the ESPRIT trial.

From the comparison of LOVAZA® versus AMR101 data, there appears to be a benefit of using pure EPA concentrates for dyslipidemia treatment over omega-3 mixtures with regard to LDL-Cholesterol and non-HDL-cholesterol effects. With the NCEP ATP III guidelines placing LDL-cholesterol and non-HDL-cholesterol reduction at the top of the treatment hierarchy for patients with TG<500 mg/dL, AMR101 is clearly superior to LOVAZA® in this patient category.

In the ECLIPSE study it is found that EPANOVA™ is significantly more bioavailable than LOVAZA® after single dose administration (four capsules of 1 gram for both products), both by Cmax (maximum concentration) and AUC (area under curve) measures (see Table C below, where Cmax and AUC are estimated from the data points in FIGS. 1 and 2). Relative to LOVAZA® under high fat meal conditions, EPANOVA™ is 1.17× more bioavailable by Cmax and 1.27 by AUC comparison. Under low fat meal conditions, LOVAZA® has only 15% AUC and 12% Cmax of the bioavailability versus LOVAZA® under high fat meal conditions, whereas EPANOVA™ under low fat meal conditions has 78% AUC and 53% Cmax of the bioavailability versus LOVAZA® under high fat meal conditions. EPANOVA™ under low fat meal conditions has 62% AUC and 46% Cmax of the bioavailability versus EPANOVA™ under high fat meal conditions.

TABLE C
Comparison of bioavailability of EPA + DHA in Plasma for Lovaza
(4 g) and Epanova (4 g) under high-fat and low-fat meal dosing conditions

| | LOVAZA - | | LOVAZA - | | Epanova - | | Epanova - | |
|---|---|---|---|---|---|---|---|---|
| | High Fat | | Low Fat | | High Fat | | Low Fat | |
| Cmax EPA + DHA | 385 | nmol/ml | 45 | nmol/ml | 450 | nmol/ml | 205 | nmol/ml |
| Est. AUC, 0-24 | 3080 | nmol*hr/ml | 465 | nmol*hr/ml | 3920 | nmol*hr/ml | 2415 | nmol*hr/ml |
| EPA + DHA | | | | | | | | |
| Tmax EPA + DHA | 5 | hrs | 10 | hrs | 5 | hrs | 5 | hrs |

| | | | | |
|---|---|---|---|---|
| Multiple of Lovaza- | 1.00 x | 0.15 x | 1.27 x | 0.78 x |
| HF AUC | | | | |
| Multiple of LF vs. | NA | 0.15 x Lovaza- | NA x | 0.62 x Epanova- |
| HF AUC | | HF AUC | | HF AUC |
| Multiple of Lovaza- | 1.00 x | 0.12 x | 1.17 x | 0.53 x |
| HF Cmax | | | | |
| Multiple of LF vs. | NA | 0.12 x Lovaza- | NA x | 0.46 x Epanova- |
| HF Cmax | | HF Cmax | | HF Cmax |
| Low fat meal - | NA | 1.00 x | NA | 5.19 x |
| AUC vs. Lov. | | | | |
| Low fat meal - | NA | 1.00 x | NA | 4.56 x |
| Cmax vs. Lov. | | | | |
| High fat meal - | 1.00 x | NA | 1.27 x | NA |
| AUC vs. Lov. | | | | |
| High fat meal - | 1.00 x | NA | 1.17 x | NA |
| Cmax vs. Lov. | | | | |

Omega-3 fatty acids are known to be “essential fatty acids”. There are two series of essential fatty acids (EFAs) in humans. They are termed “essential” because they cannot be synthesized de novo in mammals. These fatty acids can be interconverted within a series, but the omega-6 (n-6) series cannot be converted to the omega-3 series nor can the omega-3 (n-3) series be converted to the omega-6 series in humans. The main EFAs in the diet are linoleic acid of the omega-6 series and alpha-linolenic acid of the omega-3 series. However, to fulfill most of their biological effects these “parent” EFAs must be metabolised to the other longer chain fatty acids. Each fatty acid probably has a specific role in the body. The scientific literature suggests that particularly important in the n-6 series are dihomo-gammalinolenic acid (DGLA, 20:3-n6) and arachidonic acid (ARA, 20:4-n6), while particularly important in the n-3 series are eicosapentaenoic acid (EPA, 20:5-n3) and docosahexaenoic acid (DHA, 22:6-n3).

U.S. Pat. No. 6,479,544 describes an invention in which it is found that ARA is highly desirable rather than undesirable and it may be helpful to administer ARA in association with EPA. This invention provides pharmaceutical formulations containing eicosapentaenoic acid or any appropriate derivative (hereinafter collectively referred to as EPA) and arachidonic acid (ARA), as set out in the granted claims for this patent. ARA may be replaced by one or more of its precursors, DGLA or GLA. In this reference, the ratio of EPA to ARA is preferably between 1:1 and 20:1.

Patent application PCT/GB 2004/000242 describes the treatment or prevention of psoriasis with a formulation comprising more than 95% EPA and less than 2% DHA. In another embodiment of this invention the EPA is replaced with DPA.

Patent application PCT/NL 2006/050291 (WO/2007/058538, GB 0301701.9) describes combinations of idigestible oligosaccharides and long chain polyunsaturated fatty acids such as ARA, EPA, DA, and combinations thereof to improve intestinal barrier integrity, improving barrier function, stimulating gut maturation and/or reducing intestinal barrier permeability.

Lindeborg et al. (Prostag Leukotr Ess, 2013, 88:313-319) discloses a study evaluating postprandial metabolism of docosapentaenoic acid (DPA) and eicosapentaenoic acid (EPA) in humans.

Holub et al. (Lipids, 2011, 46:399-407) discloses a study assessing the effect of oral supplementation with docosapentaenoic acid (DPA) on levels of serum and tissue lipid classes and their fatty acid compositions in rat liver, heart, and kidney.

Given the highly beneficial efficacy and side-effect profile of omega-3 fatty acid concentrates, these compositions are increasingly popular for the treatment of patients with dyslipidemias. However, with the increased popularity of omega-3 fatty acid concentrates, there is an unmet medical need for omega-3 fatty acid containing compositions with improved bioavailability and absorption and a more optimal ratio of potency in reducing TG versus the resulting cholesterol profile. Specifically, agents with both a higher potency than AMR101/EPADEL® and lesser increase in LDL-C or further decrease in LDL-C and non-HDL-C than LOVAZA® are required.

Fasting triglyceride levels have been found to be correlated with the risk of cardiovascular diseases and conditions. For example, high fasting triglycerides levels have been associated with an increased risk of myocardial infarction. Gaziano et al. (Circulation, 1997; 96:2520-2525) discusses fasting triglyceride levels as a risk factor for coronary heart disease. Love-Osborne et al. (Pediatr Diabetes, 2006: 7:205-210) discloses the role of elevated fasting triglyceride levels in the development of type 2 diabetes mellitus.

All references cited herein are incorporated by reference in their entirety.

### SUMMARY OF THE INVENTION

The present invention provides orally administrable pharmaceutical compositions comprising fatty acids and one or more additional components selected from the group consisting of absorption enhancers and adjuvants. In some embodiments, the compositions have improved bioavailability and therapeutic profiles compared to comparable compositions comprising an equivalent or similar amount of fatty acids, either in ester form or free fatty acid form. The present invention also provides methods comprising administering these compositions.

The present invention provides orally administrable compositions comprise docosapentaenoic acid (DPA) and docosahexaenoic acid (DHA) in an amount of at least 60% of the total amount of the fatty acids present in the composition, wherein the ratio of DPA to DHA (DPA:DHA) is between about 10:1 to about 1:10, wherein the composition further comprises one or more additional components selected from the group consisting of absorption enhancers and adjuvants.

### DETAILED DESCRIPTION OF THE INVENTION

The present invention provides orally administrable pharmaceutical compositions comprising fatty acids and one or more additional components selected from the group consisting of absorption enhancers and adjuvants.

In some embodiments, the pharmaceutical compositions comprise at least 30% by weight of fatty acids or salts or derivatives thereof. In some embodiments, the pharmaceutical compositions comprise at least 30% by weight of omega-3 fatty acids and salts or derivatives thereof. Such omega-3 fatty acids include, but are not limited to: alpha-linolenic acid (C18:3-n3), moroctic acid (C18:4-n3), eicosatetraenoic acid (C20:4-n3), eicosapentaenoic acid (C20:5-n3), heneicosapentaenoic acid (C21:5-n3), docosapentaenoic acid (C22:5-n3), docosahexaenoic acid (C22:6-n3).

In some embodiments, omega-3 fatty acid comprising oil is present in amounts of at least 30% w/w based on the total weight of the solvent system in the dosage form, more preferably at least 40%, even more preferably at least 50%, and most preferably at least 60%. In certain embodiments, the amount can be at least 70%, more preferably at least 80%, more preferably at least 85% or most preferably at least 90%.

In some embodiments, the orally administrable composition comprises fatty acids, wherein at least 50% by weight of the fatty acids comprise omega-3-fatty acids, salts, esters, or derivatives thereof, wherein the omega-3 fatty acids comprise eicosapentaenoic acid (EPA; C20:5-n3) and docosapentaenoic acid (DPA; C22:5-n3), wherein the ratio of DHA to EPA (DHA:EPA) is less than 1:10, and wherein the ratio of DHA to DPA (DHA:DPA) is less than 2:1. In some embodiments, the composition comprises at least 50% omega-3 fatty acids, alternatively at least 55%, alternatively at least 60%, alternatively at least 65%, alternatively at least 70%, alternatively at least 75%, alternatively at least 80%, alternatively at least 85%, alternatively at least 95%, most preferably at least 90% omega-3 fatty acids of the total amount of fatty acids. In some embodiments, the composition comprises at least about 92% to about 99%, alternatively about 93% to about 98%, alternatively about 94% to about 98%, omega-3 fatty acids of the total amount of fatty acids. In some embodiments, the EPA and DPA are jointly present in the compositions of the present invention at between 55% and 100% of total fatty acids, alternatively between 60% and 100%, alternatively between 65% and 100%, alternatively between 70% and 100%, alternatively between 75% and 100%, alternatively between 80% and 100%, alternatively between 85% and 95%, alternatively between 85% and 97%, alternatively between 88% and 95%, alternatively between 88% and 97%, alternatively between 90% and 95%, alternatively between 90% and 97% of the total amount of fatty acids. In some embodiments, the ratio of DHA to EPA (DHA:EPA) is no more than 1:10, alternatively no more than 1:9, alternatively no more than 1:8, alternatively no more than 1:7, alternatively no more than 1:6, alternatively no more than 1:5. In some embodiments, the ratio of DHA to DPA (DHA:DPA) is no more than 2:1, alternatively no more than 1:1 of DHA:DPA, alternatively no more than 1:2 of DHA:DPA, alternatively no more than 1:3 of DHA:DPA, alternatively no more than 1:5 of DHA:DPA, alternatively no more than 1:8 of DHA:DPA, alternatively no more than 1:10 of DHA:DPA, alternatively no more than 1:15 of DHA:DPA, alternatively no more than 1:20 of DHA:DPA, alternatively no more than 1:25 of DHA:DPA, alternatively no more than 1:50 of DHA:DPA, alternatively no more than 1:75 of DHA:DPA, alternatively no more than 1:90 of DHA:DPA, alternatively no more than 1:95 of DHA:DPA, alternatively no more than 1:100 of DHA:DPA.

In some embodiments, the orally administrable fatty acid compositions comprise eicosapentaenoic acid (EPA) in an amount between about 70% to about 95% of the total amount of fatty acids and docosapentaenoic acid (DPA), wherein the composition comprises no more than 5% docosahexaenoic acid (DHA) of the total amount of fatty acids, and wherein the ratio of DHA:DPA is 1:1 or lower. In some embodiments, the orally administrable fatty acid compositions comprise EPA in an amount of about 70% to about 95% of the total amount of fatty acids, alternatively between 75% and 95%, alternatively between 90% and 95%, alternatively between 80% and 95%, alternatively between 90% and 95%, alternatively between 55% and 90%, alternatively between 60% and 90%, alternatively between 65% and 90%, alternatively between 70% and 90%, alternatively between 75% and 90%, alternatively between 80% and 90%, alternatively between 85% and 90%, alternatively between 55% and 92%, alternatively between 60% and 92%, alternatively between 65% and 92%, alternatively between 70% and 92%, alternatively between 75% and 92%, alternatively between 80% and 92%, alternatively between 85% and 92%, alternatively between 55% and 93%, alternatively between 60% and 93%, alternatively between 65% and 93%, alternatively between 70% and 93%, alternatively between 75% and 93%, alternatively between 80% and 93%, alternatively between 85% and 93%, alternatively more than 85%, alternatively more than 85%, alternatively between 85% and 95% EPA relative to the total amount of fatty acids present in the composition. In some embodiments, the compositions comprise about 80% to about 90%, alternatively about 81% to about 88%, alternatively about 82% to about 88%, alternatively about 83% to about 87%, alternatively about 84% to about 86%, alternatively about 85% EPA relative to the total amount of fatty acids present in the composition. In some embodiments, the orally administrable composition comprise. In some embodiments, the compositions comprise about 750 mg/g to about 950 mg/g, alternatively about 800 mg/g to about 900 mg/g, alternatively about 830 mg/g to about 870 mg/g, alternatively about 840 mg/g to about 870 mg/g, alternatively 845 mg/g to about 865 mg/g, alternatively 846 mg/g to about 860 mg/g, alternatively 847 mg/g to about 859 mg/g, alternatively about 848 mg/g to about 858 mg/g, alternatively about 849 mg/g to about 857 mg/g, alternatively about 850 mg/g to about 856 mg/g, alternatively about 851 mg/g to about 855 mg/g, alternatively about 852 mg/g to about 854 mg/g, alternatively about 853 mg/g EPA.

In some embodiments, the orally administrable composition comprises docosapentaenoic acid (DPA) in an amount of at least about 20 mg/day, alternatively at least about 30 mg/day, alternatively at least about 40 mg/day, alternatively at least about 50 mg/day, alternatively at least about 60 mg/day alternatively, at least about 70 mg/day alternatively at least about 75 mg/day, alternatively at least about 80 mg/day, alternatively at least about 90 mg/day, alternatively at least about 100 mg/day, alternatively at least about 120 mg/day, alternatively at least about 150 mg/day, alternatively at least about 200 mg/day, alternatively at least about 300 mg/day, or alternatively at least about 400 mg/day. In some embodiments, the orally administrable composition comprises at least about 45% docosapentaenoic acid (DPA) or at least about 50% or at least about 55% or at least about 60% or at least about 65% or at least about 70% or at least about 75% or at least about 80% or at least about 85% or at least about 90% or at least about 95% of DPA relative to the total amount of fatty acids present in the composition. In some embodiments, the orally administrable composition comprises no more than about 20% docosahexaenoic acid (DHA) or no more than about 15% or no more than about 12% or no more than about 10% or no more than about 8% or no more than about 7% or no more than about 6% or no more than about 5% or no more than about 4% or no more than about 3% or no more than about 2% or no more than about 1% DHA relative to the total amount of fatty acids present in the composition. In some embodiments, these orally administrable compositions comprise docosapentaenoic acid (DPA) in a significant or higher relative amount as compared to docosahexaenoic acid (DHA) such that the DPA:DHA ratio in the composition is 1:2 or greater. In some alternative embodiments, the ratio of DPA:DHA in the composition is at least 1:1, or at least 2:1 or at least 3:1, or at least 4:1 or at least 5:1.

In some embodiments, the orally administrable compositions comprise docosapentaenoic acid (DPA) and docosahexaenoic acid (DHA) in an amount of at least 60% of the total amount of the fatty acids present in the composition, wherein the ratio of DPA to DHA (DPA:DHA) is between about 10:1 to about 1:10. In some embodiments, the composition comprises DPA and DHA in an amount of about 65% or greater, alternatively about 70% or greater, alternatively about 75% or greater, alternatively about 80% or greater, alternatively about 80% or greater, alternatively about 81% or greater, alternatively about 82% or greater, alternatively about 83% or greater, alternatively about 84% or greater, alternatively about 85% or greater, alternatively about 86% or greater, alternatively about 87% or greater, alternatively about 88% or greater, alternatively about 89% or greater, alternatively about 90% or greater, alternatively about 91% or greater, alternatively about 92% or greater, alternatively about 93% or greater, alternatively about 94% or greater, alternatively about 95% or greater, alternatively about 96% or greater, alternatively about 97% or greater, alternatively about 98% or greater, or alternatively about 99% or greater of the total amount of fatty acids present in the composition. In some embodiments, the pharmaceutical composition comprises DPA and DHA in an amount of between about 70% to about 99%, alternatively about 75% to about 99%, alternatively about 80% to about 95%, of the total amount of the fatty acids present in the composition. In some embodiments, these compositions comprise DPA and DHA in a ratio of DPA to DHA (DPA:DHA) of between about 10:1 to about 1:10, alternatively about 9:1 to about 1:9, alternatively about 8:1 to 1:8, alternatively about 7:1 to about 1:7, alternatively about 6:1 to about 1:6, alternatively about 5:1 to about 1:5, or alternatively about 4:1 to about 1:4. In some embodiments, the composition comprises DPA and DHA in a ratio of DPA:DHA of at least 2:1. In some embodiments, the composition comprises DPA:DHA in a ratio of about 2:1 to about 10:1, alternatively about 2:1 to about 9:1, alternatively about 2:1 to about 8:1, alternatively about 2:1 to about 7:1, alternatively about 2:1 to about 6:1, alternatively about 2:1 to about 5:1, alternatively about 2:1 to about 4:1, alternatively about 3:1 to about 5:1, or alternatively about 4:1. In some embodiments, the composition comprises DPA and DHA in a ratio of DPA:DHA of about 5:1 to about 1:5, alternatively about 4:1 to about 1:4, alternatively about 3:1 to about 1:3, alternatively about 2:1 to about 1:2, or alternatively about 1:1.

In some embodiments, the composition comprises DHA:DPA in a ratio of about 2:1 to about 10:1, alternatively about 2:1 to about 9:1, alternatively about 2:1 to about 8:1, alternatively about 2:1 to about 7:1, alternatively about 2:1 to about 6:1, alternatively about 2:1 to about 5:1, alternatively about 2:1 to about 4:1, alternatively about 3:1 to about 5:1, or alternatively about 4:1.

In other embodiments, a relatively small amount of DHA as compared to DPA is present. In these embodiments, the compositions of the present invention comprise no more than 15:1 of DHA:DPA, alternatively no more than 12:1 of DHA:DPA, alternatively no more than 10:1 of DHA:DPA, alternatively no more than 8:1 of DHA:DPA, alternatively no more than 5:1 of DHA:DPA, alternatively no more than 3:1 of DHA:DPA, alternatively no more than 2:1 of DHA:DPA, alternatively no more than 1:1 of DHA:DPA, alternatively no more than 1:2 of DHA:DPA, alternatively no more than 1:3 of DHA:DPA, alternatively no more than 1:4 of DHA:DPA, alternatively no more than 1:5 of DHA:DPA, alternatively no more than 1:6 of DHA:DPA, alternatively no more than 1:7 of DHA:DPA, alternatively no more than 1:8 of DHA:DPA, alternatively no more than 1:10 of DHA:DPA, alternatively no more than 1:12 of DHA:DPA, alternatively no more than 1:15 of DHA:DPA, alternatively no more than 1:20 of DHA:DPA, alternatively no more than 1:25 of DHA:DPA, alternatively no more than 1:50 of DHA:DPA, alternatively no more than 1:75 of DHA:DPA, alternatively no more than 1:90 of DHA:DPA, alternatively no more than 1:95 of DHA:DPA, alternatively no more than 1:100 of DHA:DPA. In some embodiments, the ratio of DHA:DPA is less than 2:1.

The docosapentaenoic acid (DPA) may be administered in an orally administrable composition comprising DPA. In some embodiments, the compositions comprise DPA in an amount between 1% and 99% relative to the total amount of fatty acids present in the composition, alternatively between 1% and 95%, alternatively between 1% and 90%, alternatively between 1% and 85%, alternatively between 1% and 80%, alternatively between 1% and 75%, alternatively between 1% and 70%, alternatively between 1% and 65%, alternatively between 1% and 60%, alternatively between 1% and 55%, alternatively between 1% and 50%, alternatively between 1% and 45%, alternatively between 1% and 40%, alternatively between 1% and 35%, alternatively between 1% and 30%, alternatively between 1% and 25%, alternatively between 1% and 20%, alternatively between 1% and 15%, alternatively between 1% and 10%, alternatively between 1% and 5%, alternatively between 2% and 99%, alternatively between 2% and 95%, alternatively between 2% and 90%, alternatively between 2% and 85%, alternatively between 2% and 80%, alternatively between 2% and 75%, alternatively between 2% and 70%, alternatively between 2% and 65%, alternatively between 2% and 60%, alternatively between 2% and 55%, alternatively between 2% and 50%, alternatively between 2% and 45%, alternatively between 2% and 40%, alternatively between 2% and 35%, alternatively between 2% and 30%, alternatively between 2% and 25%, alternatively between 2% and 20%, alternatively between 2% and 15%, alternatively between 2% and 10%, alternatively between 2% and 5%, alternatively between 3% and 99%, alternatively between 3% and 95%, alternatively between 3% and 90%, alternatively between 3% and 85%, alternatively between 3% and 80%, alternatively between 3% and 75%, alternatively between 3% and 70%, alternatively between 3% and 65%, alternatively between 3% and 60%, alternatively between 3% and 55%, alternatively between 3% and 50%, alternatively between 3% and 45%, alternatively between 3% and 40%, alternatively between 3% and 35%, alternatively between 3% and 30%, alternatively between 3% and 25%, alternatively between 3% and 20%, alternatively between 3% and 15%, alternatively between 3% and 10%, alternatively between 3% and 5%, alternatively between 4% and 99%, alternatively between 4% and 95%, alternatively between 4% and 90%, alternatively between 4% and 85%, alternatively between 4% and 80%, alternatively between 4% and 75%, alternatively between 4% and 70%, alternatively between 4% and 65%, alternatively between 4% and 60%, alternatively between 4% and 55%, alternatively between 4% and 50%, alternatively between 4% and 45%, alternatively between 4% and 40%, alternatively between 4% and 35%, alternatively between 4% and 30%, alternatively between 4% and 25%, alternatively between 4% and 20%, alternatively between 4% and 15%, alternatively between 4% and 10%, alternatively between 4% and 5%, alternatively between 5% and 99%, alternatively between 5% and 95%, alternatively between 5% and 90%, alternatively between 5% and 85%, alternatively between 5% and 80%, alternatively between 5% and 75%, alternatively between 5% and 70%, alternatively between 5% and 65%, alternatively between 5% and 60%, alternatively between 5% and 55%, alternatively between 5% and 50%, alternatively between 5% and 45%, alternatively between 5% and 40%, alternatively between 5% and 35%, alternatively between 5% and 30%, alternatively between 5% and 25%, alternatively between 5% and 20%, alternatively between 5% and 15%, alternatively between 5% and 12%, alternatively between 5% and 10%, alternatively between 6% and 99%, alternatively between 6% and 95%, alternatively between 6% and 90%, alternatively between 6% and 85%, alternatively between 6% and 80%, alternatively between 6% and 75%, alternatively between 6% and 70%, alternatively between 6% and 65%, alternatively between 6% and 60%, alternatively between 6% and 55%, alternatively between 6% and 50%, alternatively between 6% and 45%, alternatively between 6% and 40%, alternatively between 6% and 35%, alternatively between 6% and 30%, alternatively between 6% and 25%, alternatively between 6% and 20%, alternatively between 6% and 15%, alternatively between 6% and 12%, alternatively between 6% and 11%, alternatively between 6% and 10%, alternatively between 7% and 99%, alternatively between 7% and 95%, alternatively between 7% and 90%, alternatively between 7% and 85%, alternatively between 7% and 80%, alternatively between 7% and 75%, alternatively between 7% and 70%, alternatively between 7% and 65%, alternatively between 7% and 60%, alternatively between 7% and 55%, alternatively between 7% and 50%, alternatively between 7% and 45%, alternatively between 7% and 40%, alternatively between 7% and 35%, alternatively between 7% and 30%, alternatively between 7% and 25%, alternatively between 7% and 20%, alternatively between 7% and 15%, alternatively between 7% and 12%, alternatively between 7% and 11%, alternatively between 7% and 10%, alternatively between 8% and 99%, alternatively between 8% and 95%, alternatively between 8% and 90%, alternatively between 8% and 85%, alternatively between 8% and 80%, alternatively between 8% and 75%, alternatively between 8% and 70%, alternatively between 8% and 65%, alternatively between 8% and 60%, alternatively between 8% and 55%, alternatively between 8% and 50%, alternatively between 8% and 45%, alternatively between 8% and 40%, alternatively between 8% and 35%, alternatively between 8% and 30%, alternatively between 8% and 25%, alternatively between 8% and 20%, alternatively between 8% and 15%, alternatively between 8% and 12%, alternatively between 9% and 95%, alternatively between 9% and 90%, alternatively between 9% and 85%, alternatively between 9% and 80%, alternatively between 9% and 75%, alternatively between 9% and 70%, alternatively between 9% and 65%, alternatively between 9% and 60%, alternatively between 9% and 55%, alternatively between 9% and 50%, alternatively between 9% and 45%, alternatively between 9% and 40%, alternatively between 9% and 35%, alternatively between 9% and 30%, alternatively between 9% and 25%, alternatively between 9% and 20%, alternatively between 9% and 15%, alternatively between 9% and 12%, relative to the total amount of fatty acids present in the composition. In some embodiments, the compositions comprise DPA in an amount of at least about 45% of DPA. In some alternative embodiments, the composition comprises at least 45% or at least 50% or at least 55% or at least 60% or at least 65% or at least 70% or at least 75% or at least 80% or at least 85% or at least 90% or at least 95% of DPA. In some embodiments, the composition comprises at least 20 mg of DPA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DPA. In some embodiments, the composition comprises DPA in ester form or in free fatty acid form.

The methods of treatment provides a dose of at least 20 mg DPA-N3, alternatively at least 30 mg DPA-N3, alternatively at least 40 mg DPA-N3, alternatively at least 50 mg DPA-N3, alternatively at least 60 mg DPA-N3 per day, alternatively at least 70 mg DPA-N3 per day, alternatively at least 75 mg DPA-N3 per day, alternatively at least 80 mg DPA-N3 per day, alternatively at least 90 mg DPA-N3 per day, alternatively at least 100 mg DPA-N3 per day, alternatively at least 120 mg DPA-N3 per day, alternatively at least 150 mg DPA-N3 per day, alternatively at least 160 mg DPA-N3 per day, alternatively at least 180 mg DPA-N3 per day, alternatively at least 200 mg DPA-N3 per day, alternatively at least 250 mg DPA-N3 per day, alternatively at least 300 mg DPA-N3 per day, alternatively at least 350 mg DPA-N3 per day, alternatively at least 400 mg DPA-N3 per day, alternatively at least 500 mg DPA-N3 per day, alternatively at least 600 mg DPA-N3 per day, alternatively at least 800 mg DPA-N3 or its glycerol or ethyl esters per day.

In some embodiments, the method of treatment provides a daily dose of at least about at least 20 mg of DPA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DPA. In some embodiments, the method of treatment provides a daily dose of at least about 1,000 mg DPA-N3 per day, alternatively at least about 1,500 mg DPA-N3 per day, alternatively at least about 2,000 mg DPA-N3 per day, alternatively at least about 2,500 mg DPA-N3 per day, alternatively at least about 3,000 mg DPA-N3 per day, alternatively at least about 3,500 mg DPA-N3 per day, alternatively at least about 3,750 mg DPA-N3 per day, alternatively at least about 4,000 mg DPA-N3 per day, alternatively at least about 4,250 mg DPA-N3 per day.

In some embodiments, the method of treatment provides a dose of at least about 1 mg/kg of DPA-N3 per day, alternatively about 2 mg/kg of DPA-N3 per day, alternatively about 3 mg/kg of DPA-N3 per day, alternatively about 4 mg/kg of DPA-N3 per day, alternatively about 6 mg/kg of DPA-N3 per day, alternatively about 8 mg/kg of DPA-N3 per day, alternatively about 10 mg/kg of DPA-N3 per day, alternatively about 20 mg/kg of DPA-N3 per day, alternatively about 30 mg/kg of DPA-N3 per day, and alternatively about 40 mg/kg alternatively about 50 mg/kg of DPA-N3 per day, alternatively about 75 mg/kg of DPA-N3 per day, and alternatively about 100 mg/kg.

In some embodiments, the method of treatment provides a dose of about 0 mg/kg to 1 mg/kg of DPA-N3 per day, alternatively about 1 mg/kg to 2 mg/kg of DPA-N3 per day, alternatively about 2 mg/kg to 3 mg/kg of DPA-N3 per day, alternatively about 3 mg/kg to 4 mg/kg of DPA-N3 per day, alternatively about 4 mg/kg to 6 mg/kg of DPA-N3 per day, alternatively about 6 mg/kg to 8 mg/kg of DPA-N3 per day, alternatively about 8 mg/kg to 10 mg/kg of DPA-N3 per day, alternatively about 10 mg/kg to 20 mg/kg of DPA-N3 per day, alternatively about 20 mg/kg to 30 mg/kg of DPA-N3 per day, and alternatively about 30 mg/kg to 40 mg/kg alternatively about 40 mg/kg to 50 mg/kg of DPA-N3 per day, alternatively about 50 mg/kg to 75 mg/kg of DPA-N3 per day, and alternatively about 75 mg/kg to 100 mg/kg.

The docosahexaenoic acid (DHA) may be administered in an orally administrable composition comprising DHA. In some embodiments, the compositions comprise DHA in an amount between 1% and 99% relative to the total amount of fatty acids present in the composition, alternatively between 1% and 95%, alternatively between 1% and 90%, alternatively between 1% and 85%, alternatively between 1% and 80%, alternatively between 1% and 75%, alternatively between 1% and 70%, alternatively between 1% and 65%, alternatively between 1% and 60%, alternatively between 1% and 55%, alternatively between 1% and 50%, alternatively between 1% and 45%, alternatively between 1% and 40%, alternatively between 1% and 35%, alternatively between 1% and 30%, alternatively between 1% and 25%, alternatively between 1% and 20%, alternatively between 1% and 15%, alternatively between 1% and 10%, alternatively between 1% and 5%, alternatively between 2% and 99%, alternatively between 2% and 95%, alternatively between 2% and 90%, alternatively between 2% and 85%, alternatively between 2% and 80%, alternatively between 2% and 75%, alternatively between 2% and 70%, alternatively between 2% and 65%, alternatively between 2% and 60%, alternatively between 2% and 55%, alternatively between 2% and 50%, alternatively between 2% and 45%, alternatively between 2% and 40%, alternatively between 2% and 35%, alternatively between 2% and 30%, alternatively between 2% and 25%, alternatively between 2% and 20%, alternatively between 2% and 15%, alternatively between 2% and 10%, alternatively between 2% and 5%, alternatively between 3% and 99%, alternatively between 3% and 95%, alternatively between 3% and 90%, alternatively between 3% and 85%, alternatively between 3% and 80%, alternatively between 3% and 75%, alternatively between 3% and 70%, alternatively between 3% and 65%, alternatively between 3% and 60%, alternatively between 3% and 55%, alternatively between 3% and 50%, alternatively between 3% and 45%, alternatively between 3% and 40%, alternatively between 3% and 35%, alternatively between 3% and 30%, alternatively between 3% and 25%, alternatively between 3% and 20%, alternatively between 3% and 15%, alternatively between 3% and 10%, alternatively between 3% and 5%, alternatively between 4% and 99%, alternatively between 4% and 95%, alternatively between 4% and 90%, alternatively between 4% and 85%, alternatively between 4% and 80%, alternatively between 4% and 75%, alternatively between 4% and 70%, alternatively between 4% and 65%, alternatively between 4% and 60%, alternatively between 4% and 55%, alternatively between 4% and 50%, alternatively between 4% and 45%, alternatively between 4% and 40%, alternatively between 4% and 35%, alternatively between 4% and 30%, alternatively between 4% and 25%, alternatively between 4% and 20%, alternatively between 4% and 15%, alternatively between 4% and 10%, alternatively between 4% and 5%, alternatively between 5% and 99%, alternatively between 5% and 95%, alternatively between 5% and 90%, alternatively between 5% and 85%, alternatively between 5% and 80%, alternatively between 5% and 75%, alternatively between 5% and 70%, alternatively between 5% and 65%, alternatively between 5% and 60%, alternatively between 5% and 55%, alternatively between 5% and 50%, alternatively between 5% and 45%, alternatively between 5% and 40%, alternatively between 5% and 35%, alternatively between 5% and 30%, alternatively between 5% and 25%, alternatively between 5% and 20%, alternatively between 5% and 15%, alternatively between 5% and 12%, alternatively between 5% and 10%, alternatively between 6% and 99%, alternatively between 6% and 95%, alternatively between 6% and 90%, alternatively between 6% and 85%, alternatively between 6% and 80%, alternatively between 6% and 75%, alternatively between 6% and 70%, alternatively between 6% and 65%, alternatively between 6% and 60%, alternatively between 6% and 55%, alternatively between 6% and 50%, alternatively between 6% and 45%, alternatively between 6% and 40%, alternatively between 6% and 35%, alternatively between 6% and 30%, alternatively between 6% and 25%, alternatively between 6% and 20%, alternatively between 6% and 15%, alternatively between 6% and 12%, alternatively between 6% and 11%, alternatively between 6% and 10%, alternatively between 7% and 99%, alternatively between 7% and 95%, alternatively between 7% and 90%, alternatively between 7% and 85%, alternatively between 7% and 80%, alternatively between 7% and 75%, alternatively between 7% and 70%, alternatively between 7% and 65%, alternatively between 7% and 60%, alternatively between 7% and 55%, alternatively between 7% and 50%, alternatively between 7% and 45%, alternatively between 7% and 40%, alternatively between 7% and 35%, alternatively between 7% and 30%, alternatively between 7% and 25%, alternatively between 7% and 20%, alternatively between 7% and 15%, alternatively between 7% and 12%, alternatively between 7% and 11%, alternatively between 7% and 10%, alternatively between 8% and 99%, alternatively between 8% and 95%, alternatively between 8% and 90%, alternatively between 8% and 85%, alternatively between 8% and 80%, alternatively between 8% and 75%, alternatively between 8% and 70%, alternatively between 8% and 65%, alternatively between 8% and 60%, alternatively between 8% and 55%, alternatively between 8% and 50%, alternatively between 8% and 45%, alternatively between 8% and 40%, alternatively between 8% and 35%, alternatively between 8% and 30%, alternatively between 8% and 25%, alternatively between 8% and 20%, alternatively between 8% and 15%, alternatively between 8% and 12%, alternatively between 9% and 95%, alternatively between 9% and 90%, alternatively between 9% and 85%, alternatively between 9% and 80%, alternatively between 9% and 75%, alternatively between 9% and 70%, alternatively between 9% and 65%, alternatively between 9% and 60%, alternatively between 9% and 55%, alternatively between 9% and 50%, alternatively between 9% and 45%, alternatively between 9% and 40%, alternatively between 9% and 35%, alternatively between 9% and 30%, alternatively between 9% and 25%, alternatively between 9% and 20%, alternatively between 9% and 15%, alternatively between 9% and 12%, relative to the total amount of fatty acids present in the composition. In some embodiments, the compositions comprise DHA in an amount of at least about 45% of DHA. In some alternative embodiments, the composition comprises at least 45% or at least 50% or at least 55% or at least 60% or at least 65% or at least 70% or at least 75% or at least 80% or at least 85% or at least 90% or at least 95% of DHA. In some embodiments, the composition comprises at least 20 mg of DHA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DHA. In some embodiments, the composition comprises DHA in ester form or in free fatty acid form.

The methods of treatment provides a dose of at least 20 mg DHA-N3, alternatively at least 30 mg DHA-N3, alternatively at least 40 mg DHA-N3, alternatively at least 50 mg DHA-N3, alternatively at least 60 mg DHA-N3 per day, alternatively at least 70 mg DHA-N3 per day, alternatively at least 75 mg DHA-N3 per day, alternatively at least 80 mg DHA-N3 per day, alternatively at least 90 mg DHA-N3 per day, alternatively at least 100 mg DHA-N3 per day, alternatively at least 120 mg DHA-N3 per day, alternatively at least 150 mg DHA-N3 per day, alternatively at least 160 mg DHA-N3 per day, alternatively at least 180 mg DHA-N3 per day, alternatively at least 200 mg DHA-N3 per day, alternatively at least 250 mg DHA-N3 per day, alternatively at least 300 mg DHA-N3 per day, alternatively at least 350 mg DHA-N3 per day, alternatively at least 400 mg DHA-N3 per day, alternatively at least 500 mg DHA-N3 per day, alternatively at least 600 mg DHA-N3 per day, alternatively at least 800 mg DHA-N3 or its glycerol or ethyl esters per day.

In some embodiments, the method of treatment provides a daily dose of at least about at least 20 mg of DHA, alternatively at least 30 mg, alternatively at least 40 mg, alternatively at least 50 mg, alternatively at least 60 mg, alternatively at least 90 mg, alternatively at least 100 mg, alternatively at least 120 mg, alternatively at least 150 mg, alternatively at least 200 mg, alternatively at least 300 mg, alternatively at least 400 mg of DHA. In some embodiments, the method of treatment provides a daily dose of at least about 1,000 mg DHA-N3 per day, alternatively at least about 1,500 mg DHA-N3 per day, alternatively at least about 2,000 mg DHA-N3 per day, alternatively at least about 2,500 mg DHA-N3 per day, alternatively at least about 3,000 mg DHA-N3 per day, alternatively at least about 3,500 mg DHA-N3 per day, alternatively at least about 3,750 mg DHA-N3 per day, alternatively at least about 4,000 mg DHA-N3 per day, alternatively at least about 4,250 mg DHA-N3 per day.

In some embodiments, the method of treatment provides a dose of at least about 1 mg/kg of DHA-N3 per day, alternatively about 2 mg/kg of DHA-N3 per day, alternatively about 3 mg/kg of DHA-N3 per day, alternatively about 4 mg/kg of DHA-N3 per day, alternatively about 6 mg/kg of DHA-N3 per day, alternatively about 8 mg/kg of DHA-N3 per day, alternatively about 10 mg/kg of DHA-N3 per day, alternatively about 20 mg/kg of DHA-N3 per day, alternatively about 30 mg/kg of DHA-N3 per day, and alternatively about 40 mg/kg alternatively about 50 mg/kg of DHA-N3 per day, alternatively about 75 mg/kg of DHA-N3 per day, and alternatively about 100 mg/kg.

In some embodiments, the method of treatment provides a dose of about 0 mg/kg to 1 mg/kg of DHA-N3 per day, alternatively about 1 mg/kg to 2 mg/kg of DHA-N3 per day, alternatively about 2 mg/kg to 3 mg/kg of DHA-N3 per day, alternatively about 3 mg/kg to 4 mg/kg of DHA-N3 per day, alternatively about 4 mg/kg to 6 mg/kg of DHA-N3 per day, alternatively about 6 mg/kg to 8 mg/kg of DHA-N3 per day, alternatively about 8 mg/kg to 10 mg/kg of DHA-N3 per day, alternatively about 10 mg/kg to 20 mg/kg of DHA-N3 per day, alternatively about 20 mg/kg to 30 mg/kg of DHA-N3 per day, and alternatively about 30 mg/kg to 40 mg/kg alternatively about 40 mg/kg to 50 mg/kg of DHA-N3 per day, alternatively about 50 mg/kg to 75 mg/kg of DHA-N3 per day, and alternatively about 75 mg/kg to 100 mg/kg.

The present invention also provides an administrable composition comprising fatty acids, wherein at least 50% by weight of the fatty acids comprise omega-3-fatty acids, salts, esters, or derivatives thereof, wherein the omega-3 fatty acids comprise eicosapentaenoic acid (EPA; C20:5-n3), docosapentaenoic acid (DPA; C22:5-n3), and docosahexaenoic acid (DHA; C22:6-n3), wherein the ratio of DHA to EPA (DHA:EPA) is less than 1:20, and wherein the ratio of DHA to DPA (DHA:DPA) is less than 2:1.

In some embodiments, the compositions of the present invention comprise at least 50% omega-3 fatty acids, alternatively at least 55%, alternatively at least 60%, alternatively at least 65%, alternatively at least 70%, alternatively at least 75%, alternatively at least 80%, alternatively at least 85%, alternatively at least 95%, most preferably at least 90% omega-3 fatty acids of the total amount of fatty acids.

In other embodiments, EPA and DPA are jointly present in the compositions of the present invention at between 55% and 100% of total fatty acids, alternatively between 60% and 100%, alternatively between 65% and 100%, alternatively between 70% and 100%, alternatively between 75% and 100%, alternatively between 80% and 100%, alternatively between 85% and 95%, alternatively between 85% and 97%, alternatively between 88% and 95%, alternatively between 88% and 97%, alternatively between 90% and 95%, alternatively between 90% and 97% of the total amount of fatty acids.

In other embodiments, EPA and DHA are jointly present in the compositions of the present invention at between 55% and 100% of total fatty acids, alternatively between 60% and 100%, alternatively between 65% and 100%, alternatively between 70% and 100%, alternatively between 75% and 100%, alternatively between 80% and 100%, alternatively between 85% and 95%, alternatively between 85% and 97%, alternatively between 88% and 95%, alternatively between 88% and 97%, alternatively between 90% and 95%, alternatively between 90% and 97% of the total amount of fatty acids.

In some embodiments, the compositions of the present invention comprise at least 0.01% HPA of total fatty acids in the composition, alternatively at least 0.05% HPA, alternatively at least 0.10% HPA, alternatively at least 0.15% HPA, alternatively at least 0.2% HPA, alternatively at least 0.3% HPA, alternatively at least 0.4% HPA, alternatively at least 0.5% HPA, alternatively at least 0.75% HPA, alternatively at least 1% HPA, alternatively at least 1.5% HPA, alternatively at least 2% HPA, alternatively at least 2.5% HPA, alternatively at least 3% HPA, alternatively at least 3.5% HPA, alternatively at least 4% HPA, alternatively at least 4.5% HPA, alternatively at least 5% HPA, alternatively at least 6% HPA, alternatively at least 7% HPA, alternatively the compositions of the present invention comprise at least 9% HPA of total fatty acids in the composition.

In some embodiments, the compositions of the present invention comprise no more than 20% HPA of total fatty acids in the composition, alternatively no more than 15% HPA, alternatively no more than 12% HPA, alternatively no more than 10% HPA, alternatively no more than 8% HPA, alternatively no more than 7% HPA, alternatively no more than 6% HPA, alternatively no more than 5% HPA, alternatively no more than 4% HPA, alternatively no more than 3% HPA, alternatively no more than 2% HPA, alternatively no more than 1.5% HPA, alternatively the compositions of the present invention comprise at least 1% HPA of total fatty acids in the composition. In some embodiments, the compositions of the present invention comprise 1% to 20% of the total fatty acids in the composition.

In the embodiments of the present invention, the compositions comprise EPA and DPA in an EPA:DPA ratio between 99:1 and 1:99 EPA:DPA, alternatively between 90:1 and 1:90, alternatively between 60:1 and 1:60, alternatively between 60:1 and 1:20, alternatively between 60:1 and 1:4, alternatively between 40:1 and 1:20, alternatively between 30:1 and 1:20, alternatively between 30:1 and 1:10, alternatively between 30:1 and 1:5, alternatively between 40:1 and 1:4, alternatively between 30:1 and 1:4, alternatively between 30:1 and 1:2, alternatively between 30:1 and 1:1, alternatively between 30:1 and 2:1, alternatively between 30:1 and 5:1, alternatively between 20:1 and 1:20, alternatively between 20:1 and 1:10, alternatively between 20:1 and 1:5, alternatively between 20:1 and 1:2, alternatively between 20:1 and 1:1, alternatively between 20:1 and 2:1, alternatively between 20:1 and 5:1, alternatively between 20:1 and 10:1, alternatively between 20:1 and 10:1, alternatively between 30:1 and 10:1, alternatively between 60:1 and 10:1, alternatively comprise EPA and DPA in an EPA:DPA ratio between 40:1 and 10:1.

In some embodiments, the ratio of EPA:DPA is greater than 1:1, preferably greater than 2:1, and more preferably greater than 5:1. In some embodiments, the ratio of EPA:DPA is 1:1 to 25:1, preferably 5:1 to 20:1, more preferably 8:1 to 15:1, even more preferably 9:1 to 13:1, even more most preferably about 10:1 to 11:1, and most preferably about 10:1.

In the embodiments of the present invention, the compositions comprise EPA and DHA in an EPA:DHA ratio between 99:1 and 1:99 EPA:DHA, alternatively between 90:1 and 1:90, alternatively between 60:1 and 1:60, alternatively between 60:1 and 1:20, alternatively between 60:1 and 1:4, alternatively between 40:1 and 1:20, alternatively between 30:1 and 1:20, alternatively between 30:1 and 1:10, alternatively between 30:1 and 1:5, alternatively between 40:1 and 1:4, alternatively between 30:1 and 1:4, alternatively between 30:1 and 1:2, alternatively between 30:1 and 1:1, alternatively between 30:1 and 2:1, alternatively between 30:1 and 5:1, alternatively between 20:1 and 1:20, alternatively between 20:1 and 1:10, alternatively between 20:1 and 1:5, alternatively between 20:1 and 1:2, alternatively between 20:1 and 1:1, alternatively between 20:1 and 2:1, alternatively between 20:1 and 5:1, alternatively between 20:1 and 10:1, alternatively between 20:1 and 10:1, alternatively between 30:1 and 10:1, alternatively between 60:1 and 10:1, alternatively comprise EPA and DHA in an EPA:DHA ratio between 40:1 and 10:1.

In some embodiments, the ratio of EPA:DHA is greater than 1:1, preferably greater than 2:1, and more preferably greater than 5:1. In some embodiments, the ratio of EPA:DHA is 1:1 to 25:1, preferably 5:1 to 20:1, more preferably 8:1 to 15:1, even more preferably 9:1 to 13:1, even more most preferably about 10:1 to 11:1, and most preferably about 10:1.

In some embodiments, a relatively small amount of DHA relative to the total amount of fatty acids present in the composition is present. In some embodiments, the compositions of the present invention comprise no more than 20% DHA, alternatively no more than 15% DHA, alternatively no more than 12% DHA, alternatively no more than 10% DHA, alternatively no more than 8% DHA, alternatively no more than 7% DHA, alternatively no more than 6% DHA, alternatively no more than 5% DHA, alternatively no more than 4% DHA, alternatively no more than 3% DHA, alternatively no more than 2% DHA, alternatively no more than 1% DHA relative to the total amount of fatty acids present in the composition.

In some embodiments, the ratio of DPA:HPA is about 250:1 to 1:1, alternatively 200:1 to 2:1, alternatively 150:1 to 3:1, alternatively 100:1 to 4:1, alternatively 50:1 to 5:1, alternatively 25:1 to 6:1, and alternatively 10:1 to 7:1. In some preferred embodiments, the ratio of DPA:HPA is about 8:1. In some embodiments, the ratio of DPA:HPA is about 3:0.

In some embodiments, the ratio of DHA:HPA is about 250:1 to 1:1, alternatively 200:1 to 2:1, alternatively 150:1 to 3:1, alternatively 100:1 to 4:1, alternatively 50:1 to 5:1, alternatively 25:1 to 6:1, and alternatively 10:1 to 7:1. In some preferred embodiments, the ratio of DHA:HPA is about 8:1. In some embodiments, the ratio of DHA:HPA is about 3:0.

In yet other embodiments, the compositions of the present invention comprise no more than 10% omega-6 fatty acids relative to the total amount of fatty acids, alternatively no more than 9%, alternatively no more than 8%, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% omega-6 fatty acids versus the total amount of fatty acids comprised by the compositions of the present invention.

Omega-6 fatty acids include, but are not limited to: linoleic acid (LA; C18:2-n6); gamma-linoleic acid (GLA; C18:3-n6); eicosadienoic acid (C20:2-n6); dihomo-gamma-linoleic acid (DGLA; C20:3-n6); arachiconic acid (ARA; C20:4-n6); and omega-6 docosapentaenoic acid (DPA; C22:5-n6).

In further embodiments, the compositions of the present invention comprise no more than 10% omega-6 fatty acids relative to the total amount of omega-3 fatty acids plus omega-6 fatty acids, alternatively no more than 9%, alternatively no more than 8%, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% omega-6 fatty acids versus the total amount of omega-3 fatty acids plus omega-6 fatty acids comprised by the compositions of the present invention.

In yet other embodiments, the compositions of the present invention comprise no more than 8% arachidonic acid (ARA; C20:4-n6) relative to the total amount of omega-3 fatty acids plus omega-6 fatty acids, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% arachidonic acid (ARA; C20:4-n6) versus the total amount of omega-3 fatty acids plus omega-6 fatty acids comprised by the compositions of the present invention.

In some embodiments, a relatively small amount of omega-3 fatty acids in aggregate other than EPA, ETA, HPA and DPA (alternatively indicated as non-EPA, non-ETA, non-HPA and non-DPA omega-3 fatty acids in aggregate) relative to the total amount of fatty acids present in the composition is present. In some embodiments, the compositions of the present invention comprise no more than 20% non-EPA, non-ETA, non-HPA and non-DPA omega-3 fatty acids, alternatively no more than 15% non-EPA, non-ETA, non-HPA and non-DPA omega-3 fatty acids, alternatively no more than 12% non-EPA, non-ETA, non-HPA and non-DPA omega-3 fatty acids, alternatively no more than 10% non-EPA, non-ETA, non-HPA and non-DPA omega-3 fatty acids, alternatively no more than 8% non-EPA, non-ETA, non-HPA and non-DPA omega-3 fatty acids, alternatively no more than 7% non-EPA, non-ETA, non-HPA and non-DPA omega-3 fatty acids, alternatively no more than 6% non-EPA, non-ETA, non-HPA and non-DPA omega-3 fatty acids, alternatively no more than 5% non-EPA, non-ETA, non-HPA and non-DPA omega-3 fatty acids, alternatively no more than 4% non-EPA, non-ETA, non-HPA and non-DPA omega-3 fatty acids, alternatively no more than 3% non-EPA, non-ETA, non-HPA and non-DPA omega-3 fatty acids, alternatively no more than 2% non-EPA, non-ETA, non-HPA and non-DPA omega-3 fatty acids, alternatively no more than 1% non-EPA, non-ETA, non-HPA and non-DPA omega-3 fatty acids in aggregate relative to the total amount of fatty acids present in the composition.

In some embodiments, a relatively small amount of the sum of ALA, SDA and DHA relative to the total amount of fatty acids present in the composition is present, while at the same time large amounts of the sum of EPA, DPA-n3, HPA and ETA are present. In some embodiments, the compositions of the present invention comprise no more than 20% of the sum of ALA, SDA and DHA, alternatively no more than 15% of the sum of ALA, SDA and DHA, alternatively no more than 12% of the sum of ALA, SDA and DHA, alternatively no more than 10% of the sum of ALA, SDA and DHA, alternatively no more than 8% of the sum of ALA, SDA and DHA, alternatively no more than 7% of the sum of ALA, SDA and DHA, alternatively no more than 6% of the sum of ALA, SDA and DHA, alternatively no more than 5% of the sum of ALA, SDA and DHA, alternatively no more than 4% of the sum of ALA, SDA and DHA, alternatively no more than 3% of the sum of ALA, SDA and DHA, alternatively no more than 2% of the sum of ALA, SDA and DHA, alternatively no more than 1% of the sum of ALA, SDA and DHA relative to the total amount of fatty acids present in the composition, while at the same time contain more than 40% the sum of EPA, DPAn-3, HPA and ETA, alternatively more than 50% the sum of EPA, DPAn-3, HPA and ETA, alternatively more than 60% the sum of EPA, DPAn-3, HPA and ETA, alternatively more than 70% the sum of EPA, DPAn-3, HPA and ETA, alternatively more than 75% the sum of EPA, DPAn-3, HPA and ETA, alternatively more than 80% the sum of EPA, DPAn-3, HPA and ETA, alternatively more than 85% the sum of EPA, DPAn-3, HPA and ETA, alternatively more than 90% the sum of EPA, DPAn-3, HPA and ETA, alternatively more than 95% the sum of EPA, DPAn-3, HPA and ETA, alternatively between 80% and 98% the sum of EPA, DPAn-3, HPA and ETA, alternatively between 80% and 96% the sum of EPA, DPAn-3, HPA and ETA, alternatively between 85% and 98% the sum of EPA, DPAn-3, HPA and ETA, alternatively between 85% and 96% the sum of EPA, DPAn-3, HPA and ETA, alternatively between 90% and 98% the sum of EPA, DPAn-3, HPA and ETA, alternatively between 90% and 97% the sum of EPA, DPAn-3, HPA and ETA, alternatively between 90% and 96% the sum of EPA, DPAn-3, HPA and ETA, alternatively between 90% and 95% the sum of EPA, DPAn-3, HPA and ETA, relative to the total amount of fatty acids present in the composition is present.

In further embodiments, the compositions of the present invention comprise no more than 8% arachidonic acid (ARA; C20:4-n6) relative to the total amount of fatty acids, alternatively no more than 7%, alternatively no more than 6%, alternatively no more than 5%, alternatively no more than 4.5%, alternatively no more than 4%, alternatively no more than 3.5%, alternatively no more than 3%, alternatively no more than 2.5%, alternatively no more than 2%, alternatively no more than 1.7%, alternatively no more than 1.5%, alternatively no more than 1.2%, alternatively no more than 1%, alternatively no more than 0.5% arachidonic acid (ARA; C20:4-n6) relative the total amount of fatty acids comprised by the compositions of the present invention.

In some embodiments, the composition of the present invention further comprises TPA at concentration of at least 0.05%. In some embodiments, the TPA concentration is about 0.01% to about 5%, alternatively about 0.05% to about 2%, alternatively about 0.1% to about 1%, alternatively about 0.2% to about 0.8%, alternatively about 0.4% to about 0.6%, alternatively about 0.5%.

In other embodiments, the compositions of the present invention comprise no more than 2.5% arachidonic acid (ARA; C20:4-n6), no more than 0.4% omega-6-docosapentaenoic acid (DPA; C22:5-n6) and no more than 0.2% gamma-linoleic acid (GLA; C18:3-n6) relative the total amount of fatty acids comprised by the compositions of the present invention.

Further embodiments provide fatty acid compositions comprising no more than 2.5% arachidonic acid (ARA; C20:4-n6), no more than 0.3% omega-6 docosapentaenoic acid (DPA; C22:5-n6) and no more than 0.1% gamma-linoleic acid (GLA; C18:3-n6) relative the total amount of fatty acids comprised by the compositions of the present invention.

In yet other embodiments, the active ingredient of the formulations of the present invention consists essentially wholly of the DPA and DHA or precursors thereof (ethyl ester, triglyceride, or any other pharmaceutically acceptable salt or derivative thereof). In that case, no large amounts (preferably less than 15%, alternatively less than 12%, alternatively less than 10%, alternatively less than 9%, alternatively less than 8%, alternatively less than 7%, alternatively less than 6%, alternatively less than 5%, alternatively less than 4%, alternatively less than 3%, alternatively less than 2%, alternatively less than 1%, alternatively less than 0.5%, alternatively less than 0.25%) of any other fatty acids are present.

The fatty acid percentage is determined on a weight/weight, mol/mol, or chromatography area percent basis relative to all fatty acids present in the composition as determined by methods such as disclosed in the European Pharmacopeia monograph for omega-3 fatty acid concentrates, European Pharmacopeia monograph for omega-3-acid ethyl esters 90%, or European Pharmacopeia monograph method 2.4.29, USP monograph for fish oil dietary supplements, USP 35 omega-3-acid ethyl esters (LOVAZA®) monograph, or any essentially equivalent methods (whether by gas chromatography, HPLC, FPLC or any other chromatographic method).

In some embodiments, the fatty acid percentage is determined not as a percentage of all fatty acids present in the composition but as a specific type of fatty acid ethyl esters as percentage of all fatty acid ethyl esters present in the composition, thus excluding from the fatty acid percentage determination such fatty acids present as, for instance: free fatty acids; mono-, di-, and tri-glycerides; or fatty acids present in phospholipids (such as phosphatidylserine or phosphatidylcholine) or polysorbates (such as Tween 80, Tween 20, or polysorbate 40).

In other embodiments, the fatty acid percentage is determined not as a percentage of all fatty acids present in the composition but as a specific type of free fatty acid as percentage of all free fatty acids present in the composition, thus excluding from the fatty acid percentage determination such fatty acids present as, for instance: fatty acid ethyl esters; mono-, di-, and tri-glycerides; or fatty acids present in phospholipids (such as phosphatidylserine or phosphatidylcholine) or polysorbates (such as Tween 80, Tween 20, or polysorbate 40).

In yet other embodiments, the fatty acid percentage is determined not as a percentage of all fatty acids present in the composition but as a specific type of glycerol fatty acid ester as percentage of all glycerol fatty acid esters present in the composition, thus excluding from the fatty acid percentage determination such fatty acids present as, for instance: fatty acid ethyl esters; free fatty acids; or fatty acids present in phospholipids (such as phosphatidylserine or phosphatidylcholine) or polysorbates (such as Tween 80, Tween 20, or polysorbate 40).

In further embodiments, the fatty acid percentage is determined not as a percentage of all fatty acids present in the composition but as di- or tri-fatty acid esters with glycerol as percentage of all glycerol di- and tri-fatty acid esters present in the composition, thus excluding from the fatty acid percentage determination such fatty acids present as, for instance: glycerol-mono-fatty acid esters; fatty acid ethyl esters; free fatty acids; or fatty acids present in phospholipids (such as phosphatidylserine or phosphatidylcholine) or polysorbates (such as Tween 80, Tween 20, or polysorbate 40).

In yet other embodiments, the fatty acid percentage is determined not as a percentage of all fatty acids present in the composition but as a tri-fatty acid esters with glycerol as percentage of all glycerol tri-fatty acid esters present in the composition, thus excluding from the fatty acid percentage determination such fatty acids present as, for instance: mono- and di-fatty acid esters of glycerol; fatty acid ethyl esters; free fatty acids; or fatty acids present in phospholipids (such as phosphatidylserine or phosphatidylcholine) or polysorbates (such as Tween 80, Tween 20, or polysorbate 40).

In some embodiments, suitable fatty-acid containing compositions include those described in PCT International Application Nos. PCT/US13/46176 (published as WO2013/192109), PCT/US13/073,714, PCT/US13/073,701, PCT/US13/75740, PCT/US13/75704, PCT/US13/75661, and PCT/US14/24712, each of which is incorporated by reference in its entirety. The present invention provides for the use of one or more absorption enhancers and/or adjuvants in these fatty acid-containing compositions.

The fatty acids comprised in the formulations of the present invention may be in any form. Forms of the fatty acids, including omega-3 fatty acids, include but are not limited to the free acids, salts, esters of any type (including ethyl ester), amides, mono-, di- or triglycerides, phospholipids or any other form which can lead to absorption and metabolization of one or more omega-3-fatty acids or the incorporation of one or more omega-3-fatty acids into body fluids, tissues or organs.

The omega-3 fatty acid comprising substance, for example oil, may be produced through any methods, such as those known in the art. Such methods may include but are not limited to: esterification, saponification, distillation, including short path distillation, urea precipitation; enzymatic conversion concentration; conventional chromatography; HPLC/FPLC, supercritical carbon dioxide extraction; supercritical carbondioxide chromatography; simulated moving bed chromatography; supercritical carbon dioxide simulated moving bed chromatography; or chemical conversion methods such as iodolactonization. Such methods are generally known to those skilled in the art of purifying and isolating omega-3 fatty acids compositions.

The EPA, DHA, DPA, and/or other fatty acids included in embodiments of the present invention may be derived from any appropriate source including plant seed oils, microbial oils from algae or fungal or marine oils from fish or other marine animals. They may be used in the form of the natural oil, if that oil meets the required purity requirements of the present invention, or may be purified to give products containing the fatty acid composition of the present invention.

The formulations of the present invention may comprise one or more absorption enhancers that can act to improve or increase absorption of the fatty acids. Absorption enhancers include any compound or substance which can act in one or more of the following ways: increase the bioavailability (for example, increase the area under the curve or AUC), increase the amount released from the composition, increase the Cmax (peak or maximum concentration), and decrease the Tmax (time to reach Cmax) of an active ingredient, such as fatty acids. Absorption enhancers may also promote dispersion and dissolution prior to passage to the systemic circulation. In some embodiments, the formulations of the present invention, which comprise one or more absorption enhancers, demonstrate superior effects compared to similar formulations not containing absorption enhancers. For example, the formulations of the present invention, which contain absorption enhancers, may provide increased bioavailability (AUC) of one or more fatty acids (preferably omega-3 fatty acids), increased amount of release of one or more fatty acids, increased Cmax of one or more fatty acids, and/or decreased Tmax of one or more fatty acids, compared to formulation containing the same or similar amount of fatty acids but not containing absorption enhancer. Examples of such formulations (not containing absorption enhancer) include, but are not limited to, LOVAZA®, EPANOVA®, and VASCEPA®.

Absorption enhancers comprised in formulations of the present invention may include, but are not limited to antioxidants and preservatives. The present invention provides formulation comprises one or more absorption enhancers. In some embodiments, the formulation comprises two or more absorption enhancers. In some embodiments, the formulation comprises three or more absorption enhancers.

The absorption enhancers may be present in: the fatty acid-comprising oil of the formulation (for example, in the omega-3 fatty acid-containing oil); in the gelatin of a soft or hard gelatin (or other gelatinous compound) capsule shell containing the omega-3 comprising oil; in a coating covering part of all of the capsule gel, or in any other compartment of a formulation comprising an omega-3 comprising oil. The absorption enhancer may be present in one part of the formulation or in more than one part of the formulation. For example, an absorption enhancer may be present in just the oil, capsule shell, or coating. Alternatively, an absorption enhancer may be present in the oil and/or the capsule shell, and/or coating. In some embodiments wherein the formulation comprises more than one absorption enhancer, one or more of the absorption enhancers may all be present in the same part of the formulation, or they may be present in different parts of the formulation. For example, all of the absorption enhancers may be present in the fatty acid-comprising oil, or the capsule shell, or in a coating on the capsule shell. Alternatively, one absorption enhancer may be present in the oil, while another absorption enhancer(s) is present in, for example, the capsule shell or coating.

Examples of absorption enhancers include, but are not limited to phenolic antioxidants, such as propyl gallate, butylated hydroxyansole (BHA), butylated hydroxytoluene (BHT), tert-butyl-hydroquinone (TBHQ), 4-hydroxymethyl-2,6-di-ter-butylphenol (HMBP), 2,4,5-trihydroxybutyrophenone (THBP), gallic acid, alpha-tocopherol and esters, derivatives, analogues or equivalents thereof. Additional examples include, but are not limited to, hexadecyldimethylbenzylammonium chloride (BAC), and hexasalicylic acid (HAS) and derivatives, analogues, and equivalents thereof.

In some embodiments, the composition of the present invention comprises propyl gallate. In some embodiments of the present invention, propyl gallate is present in the formulation, preferably in the fatty acid-containing oil or coating, in an amount up to 0.2 mg/g, more preferably up to 0.5 mg/g, more preferably up to 1 mg/g, more preferably up to 2.5 mg/g, and most preferably up to 5 mg/g. In some embodiments of the present invention, propyl gallate is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, and most preferably up to 10 mg/g.

In some embodiments, the composition of the present invention comprises butylated hydroxyanisole (BHA). In some embodiments, butylated hydroxyanisole is present in the formulation, preferably in the fatty acid-containing oil or coating, in an amount up to 0.2 mg/g, more preferably up to 0.5 mg/g, more preferably up to 1 mg/g, more preferably up to 2.5 mg/g, and most preferably up to 5 mg/g. In some embodiments, butylated hydroxyanisole is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, most preferably up to 10 mg/g.

In some embodiments, the composition of the present invention comprises butylated hydroxytoluene (BHT). In some embodiments, butylated hydroxytoluene is present in the formulation, preferably in the fatty acid-containing oil or coating, in an amount up to 0.2 mg/g, more preferably up to 0.5 mg/g, more preferably up to 1 mg/g, more preferably up to 2.5 mg/g, and most preferably up to 5 mg/g. In some embodiments, butylated hydroxytoluene is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, and most preferably up to 10 mg/g.

In some embodiments, the composition of the present invention comprises tertiary butylhydroquinone (TBHQ). In some embodiments, tertiary butylhydroquinone is present in the formulation, preferably in the fatty acid-containing oil or coating, in an amount up to 0.2 mg/g, more preferably up to 0.5 mg/g, more preferably up to 1 mg/g, more preferably up to 2.5 mg/g, and most preferably up to 5 mg/g. In some embodiments, tertiary butylhydroquinone is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, most preferably up to 10 mg/g.

In some embodiments, the composition of the present invention comprises 2,4,5-trihydroxybutyrophenone (THBP). In some embodiments, the 2,4,5-trihydroxybutyrophenone is present in the formulation, preferably in the fatty acid-containing oil or coating, in an amount up to 0.2 mg/g, more preferably up to 0.5 mg/g, more preferably up to 1 mg/g, more preferably up to 2.5 mg/g, and most preferably up to 5 mg/g. In some embodiments, 2,4,5-trihydroxybutyrophenone is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, most preferably up to 10 mg/g.

In some embodiments, the composition of the present invention comprises alpha-tocopherol or derivatives or analogues thereof. Examples of alpha-tocopherol ester, derivatives and analogues include, but are not limited to: alpha-tocopheryl ester, alpha-tocopheryl acetate, 6-hydroxy-2,5,7,8-tetramethylchroman-2-carboxylic acid (also known as Trolox), alpha-tocopheryl succinate, and alpha-tocopheryl polyethylene glycol succinate. In some embodiments, the composition comprises alpha-tocopheryl polyethylene glycol succinate. In some embodiments, alpha-tocopheryl polyethylene glycol succinate is present in the formulation, preferably in the fatty acid-containing oil or coating, in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, and most preferably up to 20 mg/g. In some embodiments, alpha-tocopheryl polyethylene glycol succinate is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, more preferably up to 20 mg/g, most preferably up to 50 mg/g.

In some embodiments, the composition of the present invention comprises hexadecyldimethylbenzylammonium chloride. In some embodiments, hexadecyldimethylbenzylammonium chloride is present in the formulation, preferably in the fatty-acid containing oil or coating, in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, and most preferably up to 20 mg/g. In some embodiments, hexadecyldimethylbenzylammonium chloride is present in the gelatin of a soft or hard gelatin (or other gelatinous compound) capsule shell containing the fatty acid-comprising oil of the formulation in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, more preferably up to 20 mg/g, most preferably up to 50 mg/g.

In some embodiments, the composition of the present invention comprises hexylsalicylic acid. In some embodiments, the hexylsalicylic acid is present in the formulation, preferably in the fatty-acid containing oil or coating, in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, and most preferably up to 20 mg/g. In some embodiments, hexylsalicylic acid is present in the gelatin of a soft or hard gelatin (or other gelatinous compound) capsule shell containing the fatty-acid comprising oil of the formulation in an amount up to 0.2 mg/g, more preferably up to 0.5 mg/g, more preferably up to 1 mg/g, more preferably up to 2.5 mg/g, more preferably up to 5 mg/g, more preferably up to 10 mg/g, more preferably up to 20 mg/g, most preferably up to 50 mg/g.

In some embodiments, the orally administrable composition comprises one or more adjuvants. In some embodiments, the orally administrable compositions of the present invention further comprise adjuvants in addition to the absorption enhancer. In some embodiments, the formulations of the present invention, which may comprise one or more adjuvants, demonstrate superior effects compared to similar formulations not containing absorption enhancers. For example, the formulations of the present invention, which may contain one or more adjuvants, may provide increased bioavailability (AUX) of one or more fatty acids (preferably omega-3 fatty acids), increased amount of release of one or more fatty acids, increased Cmax of one or more fatty acids, and/or decreased Tmax of one or more fatty acids, compared to formulation containing the same or similar amount of fatty acids but not containing adjuvants. Examples of such formulations (not containing absorption enhancer) include, but are not limited to, LOVAZA®, EPANOVA®, and VASCEPA®. These formulations of the present invention may also have improved pharmacological features and an improved therapeutic profile versus pharmaceutical dosage forms comprising almost entirely ethyl esters of omega-3 fatty acids (such as LOVAZA® and VASCEPA)®) and pharmaceutical dosage forms comprising almost entirely omega-3 free fatty acids (such as EPANOVA®).

Adjuvants include, but are not limited to surfactants, preferably non-ionic surfactants, and emulsifiers (emulsifying agents and emulsion stabilizers), such as oleic acid and salts or esters thereof. Non-ionic surfactants include, but are not limited to fatty alcohols, cetyl alcohol, stearyl alcohol, cetostearyl alcohol, and oleyl alcohol. Examples of non-ionic surfactants include but are not limited to polyoxyethylene glycol alkyl ethers, polyoxypropylene glycol alkyl ethers, glucoside alkyl ethers, polyoxyethylene glycol octylphenol ethers, glycerol alkyl esters, polyoxyethylene glycol sorbitan alkyl esters, sorbitan alkyl esters, dodecyldimethylamine oxide, block copolymers of polyethylene glycol and polypropylene glycol, and polyethoxylated tallow amine. Emulsifying agents and emulsion stabilizers (emulsifiers) include, but are not limited to, cationic emulsifying agents such as benzalkonium chloride and benzethonium chloride; anionic emulsifying agents such as sodium or potassium oleate, triethanolamine stearate, sodium lauryl sulfate, sodium dioctyl sulfosuccinate, and sodium docusate; and nonionic emulsifying agents such as sorbitan esters, polyoxyethylene derivatives of sorbitan esters, and glyceryl esters. Examples of adjuvants include oleates such as glyceryl oleate (also called glyceryl mono-oleate), sorbitan oleate (also called sorbitan mono-oleate), polyglyceryl-3-oleate, sodium oleate, ethyl oleate, or equivalents thereof. Adjuvants may also comprise polyalcohols esterified with oleic acid and carbohydrates esterified with oleic acid. The polyalcohols (also called polyhydric alcohols) include, but are not limited to, glycerol, methanol, glycol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, and polyglycitol. In some preferred embodiments, the polyalcohol comprises glycerol. Examples of carbohydrates include but are not limited to monosaccharides such as glucose, fructose and galactose; disaccharides such as sucrose, lactose, and maltose; oligosaccharides such as those containing glucose, fructose and/or galactose; and polysaccharides, such as those containing glucose, fructose, and/or galactose. In some preferred embodiments, the carbohydrates are simple carbohydrates (monosaccharides and disaccharides). In some embodiments, the adjuvant comprises an essential fatty acid, including but not limited to C₁₂, C₁₄, C₁₆, C₁₈ and C₂₀ fatty acids in mono- or oligo-unsaturated (containing 2 or 3 double bonds) fatty acid form.

In the embodiments of the present invention containing an adjuvant, the adjuvant is present in the formulation in an amount of at least 1 mg/g, more preferably at least 2 mg/g, more preferably at least 5 mg/g, more preferably at least 10 mg/g, more preferably at least 15 mg/g, more preferably at least 20 mg/g, more preferably at least 50 mg/g, most preferably at least 100 mg/g. In one embodiment, such quantification of the amount of adjuvant present applies to the total weight of the formulation or dosage form. In another embodiment, such quantification of the amount of adjuvant present applies the relative presence on a w/w basis in the fatty acid (preferably omega-3 fatty acid)-comprising oil contained in the orally administrable pharmaceutical composition.

In some embodiments, the orally administrable pharmaceutical composition of the present invention contains absorption enhancer and adjuvant and demonstrates a synergistic effect. Synergy refers to the effect wherein a combination of two or more components provides a result which is greater than the sum of the effects produced by the agents when used alone. In some embodiments, the synergistic effect is greater than an additive effect. In some embodiments, the combination of absorption enhancer and adjuvant is synergistic and formulations comprising absorption enhancer and adjuvant provide a statistically significant, greater effect compared to: (1) formulations containing absorption enhancer but no adjuvant, and/or (2) formulations containing adjuvant but no absorption enhancer. The synergistic effect may relate to increased bioavailability of one or more fatty acids, increased amount of release of one or more fatty acids, increased Cmax of one or more fatty acids, and/or decreased Tmax of one or more fatty acids. In some preferred embodiments, the fatty acids refer to omega-3 fatty acids.

The present invention provides methods of administering the above-described compositions to a subject in need thereof. The compositions of the present invention may be used for the treatment of patients by administering an effective amount of such formulations to a subject prone to or afflicted with such disease (or in need of treatment for its disease or condition), and wherein the disease/condition is an amenable disease/condition selected from the group consisting of: hypertriglyceridemia; hypercholesterolemia; mixed dyslipidemia; coronary heart disease (CHD); vascular disease; atherosclerotic disease and related conditions; heart failure; cardiac arrhythmias; ischemic dementia; hypertension; coagulation related disorders; nephropathy; kidney or urinary tract disease; retinopathy; cognitive, psychiatric, neurological and other CNS disorders, including but not limited to schizophrenia, depression, bipolar disorder and any form of dementia; autoimmune diseases; inflammatory diseases; asthma, COPD or other respiratory disease; dermatological disease; metabolic syndrome; diabetes or other forms of metabolic disease; liver diseases including fatty liver disease; diseases affecting the senses, including those affecting vision and hearing; diseases of the gastrointestinal tract; diseases of the male or female reproductive system or related secondary sexual organs; a cancer; any infections caused by a virus, bacterium, fungus, protozoa or other organism; and the treatment and/or prevention and/or reduction of cardiac events and/or cardiovascular events and/or vascular events and/or symptoms.

Cardiovascular and/or cardiac events may include, but are not limited to: myocardial infarction, ischemic cardiac attack, ischemic attack, acute angina, hospitalization due to acute angina, stroke, transient ischemic cerebral attack, cardiac revascularization, cardiac revascularization with stent placement, carotid artery revascularization, carotid artery revascularization with stent placement, peripheral artery revascularization, peripheral artery revascularization with stent placement, plaque rupture, death due to cardiovascular event, and hospitalization due to cardiovascular event.

In some embodiments, the improved pharmacological features of the formulations of the present invention may be demonstrated by improved bioavailability in a mammal of EPA, DHA, or EPA+DHA combined. Key parameters for determining bioavailability are; maximum concentration of a therapeutic compound or a metabolite thereof (Cmax); the time from administration to maximum concentration (Tmax); and the area under the concentration curve over time (AUC). Such parameters may be determined under single dose or multiple dose administration regimens. Methods to determine comparative bioavailability in mammals are generally known to those skilled in the art. When comparing Tₘₐₓ, Cmax, and AUC for embodiments of the present invention to LOVAZA®, EPANOVA®, and VASCEPA® (also known as AMR101) throughout this application, such comparison will be on the basis of an equal dose of 4 capsules of 1 gram each for each of these products. The comparative parameters, however, do apply to all essentially equivalent dosing modes comparing embodiments of the present invention to LOVAZA®, EPANOVA®, and VASCEPA®.

Meal conditions during administration to a subject of omega-3 fatty acid compositions or omega-3 fatty acid formulations are of special significance for absorption and bioavailability of omega-3 fatty acids. The meal conditions typically considered are: fasting (no food at all prior for 6-8 hours prior to administration and 2-3 hours post administration of the treatment); a low fat meat (a meal containing 5 gram to 25 gram of fat [350-600 Kcal] consumed just before or after the administration of the treatment; typically within a 15-30 minute range); or a high fat meat (a meal containing 40 gram to 75 gram of fat [700-1000 Kcal] consumed just before or after the administration of the treatment; typically within a 15-30 minute range).

In some embodiments of the present invention, formulations of the present invention are more rapidly absorbed as measured by the time to reach the maximum concentration (Tmax) in blood, serum or plasma of EPA, DHA, EPA+DHA, or total omega-3 fatty acids. In preferred embodiments of the present invention, Tmax under high fat meal administration conditions is less than 8 hours, more preferably less than 6 hours, more preferably approximately 5 hours, most preferably 4 hours or less. In other preferred embodiments of the present invention, Tmax under low fat meal administration conditions is less than 8 hours, more preferably less than 6 hours, more preferably approximately 5 hours, most preferably 4 hours or less. In yet other preferred embodiments of the present invention, Tmax under fasting administration conditions is less than 8 hours, more preferably less than 6 hours, more preferably approximately 5 hours, most preferably 4 hours or less. In further embodiments of the present invention, Tmax for EPA+DHA and total omega-3 fatty acids are equal or less than than Tmax for LOVAZA® for EPA+DHA and total omega-3 fatty acids under high fat meat, low fat meal, and fasting administration conditions. In other embodiments of the present invention, Tmax for EPA+DHA and total omega-3 fatty acids are less than Tmax for LOVAZA® for EPA+DHA and total omega-3 fatty acids under either low fat meal, fasting, or both administration conditions.

In yet other embodiments of the present invention, Tmax for EPA+DHA and total omega-3 fatty acids are equal or less than Tmax for VASCEPA® for EPA+DHA and total omega-3 fatty acids under high fat meat, low fat meal, and fasting administration conditions. Finally, in other embodiments of the present invention, Tmax for EPA+DHA and total omega-3 fatty acids are less than Tmax for VASCEPA® for EPA+DHA and total omega-3 fatty acids under either low fat meal, fasting, or both administration conditions.

In other embodiments of the present invention, formulations of the present invention are better absorbed than LOVAZA® as measured by the maximum concentration (Cmax) in blood, serum or plasma of EPA, DHA, EPA+DHA, or total omega-3 fatty acids.

In preferred embodiments of the present invention, Cmax for EPA+DHA and total omega-3 fatty acids under high fat meal administration conditions are preferably at least 1.1×(110% of) Cmax for EPA+DHA and total omega-3 fatty acids for LOVAZA®, more preferably at least 1.2×(120% of), more preferably at least 1.3×(130% of), more preferably at least 1.4×(140% of), most preferably at least 1.5×(150% of) Cmax for EPA+DHA and total omega-3 fatty acids for LOVAZA®.

In other preferred embodiments of the present invention, Cmax for EPA+DHA and total omega-3 fatty acids under low fat meal administration conditions are preferably at least 1.5×(150% of) Cmax for EPA+DHA and total omega-3 fatty acids for LOVAZA®, more preferably at least 2×(200% of), more preferably at least 3×(300% of), more preferably at least 4×(400% of), more preferably at least 5×(500% of), most preferably at least 6×(600% of) Cmax for EPA+DHA and total omega-3 fatty acids for LOVAZA®.

In other preferred embodiments of the present invention, Cmax for EPA+DHA and total omega-3 fatty acids under fasting administration conditions are preferably at least 1.5×(150% of) Cmax for EPA+DHA and total omega-3 fatty acids for LOVAZA®, more preferably at least 2×(200% of), more preferably at least 3×(300% of), more preferably at least 4×(400% of), more preferably at least 5×(500% of), more preferably at least 6×(600% of), most preferably at least 7×(700% of) Cmax for EPA+DHA and total omega-3 fatty acids for LOVAZA®.

In other embodiments of the present invention, formulations of the present invention are better absorbed than LOVAZA® as measured by the area under the concentration curve over time (AUC) in blood, serum or plasma of EPA, DHA, EPA+DHA, or total omega-3 fatty acids.

In preferred embodiments of the present invention, AUC for EPA+DHA and total omega-3 fatty acids under high fat meal administration conditions are preferably at least 1.1×(110% of) AUC for EPA+DHA and total omega-3 fatty acids for LOVAZA®, more preferably at least 1.2×(120% of), more preferably at least 1.3×(130% of), more preferably at least 1.4×(140% of), most preferably at least 1.5×(150% of) AUC for EPA+DHA and total omega-3 fatty acids for LOVAZA®.

In other preferred embodiments of the present invention, AUC for EPA+DHA and total omega-3 fatty acids under low fat meal administration conditions are preferably at least 1.5×(150% of) AUC for EPA+DHA and total omega-3 fatty acids for LOVAZA®, more preferably at least 2×(200% of), more preferably at least 3×(300% of), more preferably at least 4×(400% of), more preferably at least 5×(500% of), most preferably at least 6×(600% of) AUC for EPA+DHA and total omega-3 fatty acids for LOVAZA®.

In other preferred embodiments of the present invention, AUC for EPA+DHA and total omega-3 fatty acids under fasting administration conditions are preferably at least 1.5×(150% of) AUC for EPA+DHA and total omega-3 fatty acids for LOVAZA®, more preferably at least 2×(200% of), more preferably at least 3×(300% of), more preferably at least 4×(400% of), more preferably at least 5×(500% of), more preferably at least 6×(600% of), most preferably at least 7×(700% of) AUC for EPA+DHA and total omega-3 fatty acids for LOVAZA®.

In other embodiments of the present invention, formulations of the present invention are better absorbed than VASCEPA® as measured by the maximum concentration (Cmax) in blood, serum or plasma of EPA, DHA, EPA+DHA, or total omega-3 fatty acids.

In preferred embodiments of the present invention, Cmax for EPA+DHA and total omega-3 fatty acids under high fat meal administration conditions are preferably at least 1.1×(110% of) Cmax for EPA+DHA and total omega-3 fatty acids for VASCEPA®, more preferably at least 1.2×(120% of), more preferably at least 1.3×(130% of), more preferably at least 1.4×(140% of), most preferably at least 1.5×(150% of) Cmax for EPA+DHA and total omega-3 fatty acids for VASCEPA®.

In other preferred embodiments of the present invention, Cmax for EPA+DHA and total omega-3 fatty acids under low fat meal administration conditions are preferably at least 1.5×(150% of) Cmax for EPA+DHA and total omega-3 fatty acids for VASCEPA®, more preferably at least 2×(200% of), more preferably at least 3×(300% of), more preferably at least 4×(400% of), more preferably at least 5×(500% of), most preferably at least 6×(600% of) Cmax for EPA+DHA and total omega-3 fatty acids for VASCEPA®.

In other preferred embodiments of the present invention, Cmax for EPA+DHA and total omega-3 fatty acids under fasting administration conditions are preferably at least 1.5×(150% of) Cmax for EPA+DHA and total omega-3 fatty acids for VASCEPA®, more preferably at least 2×(200% of), more preferably at least 3×(300% of), more preferably at least 4×(400% of), more preferably at least 5×(500% of), more preferably at least 6×(600% of), most preferably at least 7×(700% of) Cmax for EPA+DHA and total omega-3 fatty acids for VASCEPA®.

In other embodiments of the present invention, formulations of the present invention are better absorbed than VASCEPA® as measured by the area under the concentration curve over time (AUC) in blood, serum or plasma of EPA, DHA, EPA+DHA, or total omega-3 fatty acids.

In preferred embodiments of the present invention, AUC for EPA+DHA and total omega-3 fatty acids under high fat meal administration conditions are preferably at least 1.1×(110% of) AUC for EPA+DHA and total omega-3 fatty acids for VASCEPA®, more preferably at least 1.2×(120% of), more preferably at least 1.3×(130% of), more preferably at least 1.4×(140% of), most preferably at least 1.5×(150% of) AUC for EPA+DHA and total omega-3 fatty acids for VASCEPA®.

In other preferred embodiments of the present invention, AUC for EPA+DHA and total omega-3 fatty acids under low fat meal administration conditions are preferably at least 1.5×(150% of) AUC for EPA+DHA and total omega-3 fatty acids for VASCEPA®, more preferably at least 2×(200% of), more preferably at least 3×(300% of), more preferably at least 4×(400% of), more preferably at least 5×(500% of), most preferably at least 6×(600% of) AUC for EPA+DHA and total omega-3 fatty acids for VASCEPA®.

In other preferred embodiments of the present invention, AUC for EPA+DHA and total omega-3 fatty acids under fasting administration conditions are preferably at least 1.5×(150% of) AUC for EPA+DHA and total omega-3 fatty acids for VASCEPA®, more preferably at least 2×(200% of), more preferably at least 3×(300% of), more preferably at least 4×(400% of), more preferably at least 5×(500% of), more preferably at least 6×(600% of), most preferably at least 7×(700% of) AUC for EPA+DHA and total omega-3 fatty acids for VASCEPA®.

In other embodiments of the present invention, formulations of the present invention are better absorbed than EPANOVA® as measured by the maximum concentration (Cmax) in blood, serum or plasma of EPA, DHA, EPA+DHA, or total omega-3 fatty acids.

In preferred embodiments of the present invention, Cmax for EPA+DHA and total omega-3 fatty acids under high fat meal administration conditions are preferably approximately 1.0×(100% of) Cmax (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) Cmax for EPA+DHA and total omega-3 fatty acids for EPANOVA®.

In other preferred embodiments of the present invention, Cmax for EPA+DHA and total omega-3 fatty acids under low fat meal administration conditions are preferably approximately 1.0×(100% of) Cmax (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) Cmax for EPA+DHA and total omega-3 fatty acids for EPANOVA®.

In other preferred embodiments of the present invention, Cmax for EPA+DHA and total omega-3 fatty acids under fasting administration conditions are preferably approximately 1.0×(100% of) Cmax (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) Cmax for EPA+DHA and total omega-3 fatty acids for EPANOVA®.

In other embodiments of the present invention, formulations of the present invention are better absorbed than EPANOVA® as measured by the area under the concentration curve over time (AUC) in blood, serum or plasma of EPA, DHA, EPA+DHA, or total omega-3 fatty acids.

In preferred embodiments of the present invention, AUC for EPA+DHA and total omega-3 fatty acids under high fat meal administration conditions are preferably approximately 1.0×(100% of) AUC (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) AUC for EPA+DHA and total omega-3 fatty acids for EPANOVA®.

In other preferred embodiments of the present invention, AUC for EPA+DHA and total omega-3 fatty acids under low fat meal administration conditions are preferably approximately 1.0×(100% of) AUC (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) AUC for EPA+DHA and total omega-3 fatty acids for EPANOVA®.

In other preferred embodiments of the present invention, AUC for EPA+DHA and total omega-3 fatty acids under fasting administration conditions are preferably approximately 1.0×(100% of) AUC (or non-significant difference) for EPA+DHA and total omega-3 fatty acids for EPANOVA®, more preferably at least 1.05×(105% of), more preferably at least 1.1×(110% of), more preferably at least 1.2×(120% of), most preferably at least 1.3×(130% of) AUC for EPA+DHA and total omega-3 fatty acids for EPANOVA®.

In some embodiments, the improved bioavailability features described above are apparent upon single dose administration, while in other embodiments the improved bioavailability features described above are apparent after multiple dose administration of formulations according to the present invention as compared to referenced comparator products above or substantial equivalent forms thereof.

In other embodiments, the improved profile of the formulations of the present invention may be demonstrated by a differentiated impact on blood/serum/plasma lipid and lipoprotein levels in a mammal; these include, but are not limited to: TG, total-cholesterol, non-HDL-cholesterol, LDL-cholesterol, VLDL-cholesterol, Apolipoprotein B, Apolipoprotein A, HDL-cholesterol, and Lp-PLA2. Methods to determine comparative blood/serum/plasma lipid and lipoprotein levels and therapeutic effects on these levels in mammals are generally know to those skilled in the art. Differences of active treatment versus placebo are generally assessed on a group of subjects versus another group of subjects basis, with significant changes noted if the p-value for the appropriate statistical comparison is equal to or less than 0.05. P-values larger than 0.05 are generally considered not significant (NS).

In one embodiment, the formulations of the present invention as compared to placebo are more potent than other omega-3 compositions known in the prior art (such as LOVAZA®, EPANOVA®, and VASCEPA®) in reducing: TG levels, Total-cholesterol levels, non-HDL-cholesterol levels, VLDL-cholesterol levels, apolipoprotein-B levels, or any combination thereof.

In another embodiment, the formulations of the present invention as compared to placebo result in minor or non-significant changes in LDL-cholesterol levels in patients with baseline TG levels above 500 mg/dL. In yet another embodiment, the formulations of the present invention as compared to placebo result in significant reductions in LDL-cholesterol levels in patients with baseline TG levels of 200-499 mg/dL while on statin therapy.

In yet another embodiment, the formulations of the present invention as compared to placebo are more potent than other omega-3 compositions known in the prior art (such as LOVAZA®, EPANOVA®, and VASCEPA®) in increasing HDL-cholesterol levels, apolipoprotein-A levels, or a combination thereof.

In yet other embodiments, the formulations of the present invention as compared to placebo are more potent than other omega-3 compositions known in the prior art (such as LOVAZA®, EPANOVA®, and VASCEPA®) in reducing TG while causing a lesser increase in LDL-cholesterol, a lesser non-significant increase in LDL-cholesterol, or no increase in LDL-cholesterol at all in patients with baseline TG levels above 500 mg/dL.

The present invention provides methods of treating a condition in a subject in need thereof. Examples of conditions include, but are not limited to: hypertriglyceridemia; hypercholesterolemia; mixed dyslipidemia; coronary heart disease (CHD); vascular disease; cardiovascular disease; acute coronary syndrome; atherosclerotic disease and related conditions; heart failure; cardiac arrhythmias; coagulatory conditions associated with cardiac arrhythmias; ischemic dementia; vascular dementia; hypertension; coagulation related disorders; nephropathy; kidney or urinary tract disease; retinopathy; cognitive and other CNS disorders; autoimmune diseases; inflammatory diseases; asthma or other respiratory disease; dermatological disease; metabolic syndrome; diabetes, diabetes mellitis or other form of metabolic disease; liver disease; non-alcoholic fatty liver disease; disease of the gastrointestinal tract; disease of the male or female reproductive system or related secondary sexual organs; a cancer of any type, including lymphomas and myelomas; and an infection caused by a virus, bacterium, fungus, protozoa or other organism.

The formulations of the present invention are also useful to treat coronary heart disease (CHD), vascular disease, atherosclerotic disease or related conditions. The compositions of the present invention may also be use for the treatment and/or prevention and/or reduction of cardiac events and/or cardiovascular events and/or vascular events and/or symptoms. Determination of such cardiovascular diseases/conditions and prevention of events/symptoms in mammals and methods to determine treatment and preventative/therapeutic effects therefore are generally know to those skilled in the art.

The present invention also relates to treatment of such conditions in with concomitant treatments regimes or combination products with other active pharmaceutical ingredients. Such concomitant or fixed combination treatments may include a statin, an anticoagulant (such as aspirin or clopidogrel), an antihypertensive (such as a diuretic, beta-blocker, calcium channel blocker, ACE-inhibitor, angiotensin II receptor (ARB) antagonist, or other treatments for cardiovascular diseases.

The present invention also includes pharmaceutical formulations, for example, a unit dosage, comprising one or more statins and the omega-3 fatty acid composition of the present invention. The present invention may incorporate now known or future known statins in an amount generally recognized as safe. There are currently seven statins that are widely available: atorvastatin, rosuvastatin, fluvastatin, lovastatin, pravastatin, pitavastatin, and simvastatin. An eight statin, cerivastatin, has been removed from the U.S. market at the time of this writing. However, it is conceivable to one skilled in the art that cerivastatin may be used in conjunction with some embodiments of the present invention if cerivastatin is ultimately determined to be safe and effective.

Generally, the effect of statins is dose dependent, i.e., the higher the dose, the greater the therapeutic effect. However, the effect of each statin is different, and therefore the level of therapeutic effect of one statin cannot be necessarily be directly correlated to the level of therapeutic effects of other statins. For example, bioavailability varies widely among the statins. Specifically, it has been shown that simvastatin is less than 5% bioavailable, while fluvastatin is approximately 24% bioavailable. Statins are absorbed at rates ranging from about 30% with lovastatin to 98% with fluvastatin. First-pass metabolism occurs in all statins except pravastatin. Pravastatin is also the least protein-bound of the statins (about 50%), compared with the others, which are more than 90% protein-bound. Accordingly, the statins possess distinct properties from one another. The combination products of this invention involving each statin or a plurality of statins are also distinct.

The formulations of the present invention may also be taken as a general nutritional supplement.

The concentrated omega-3 fatty acids can be administered in a daily amount of from about 0.1 g to about 10 g, more preferably about 0.2 g to about 8 g, and most preferably from about 0.5 g to about 4 g. Preferably, in the unit dosage form, the omega-3 fatty acids are present in an amount from about 0.1 g to about 2 g, preferably about 0.5 g to about 1.5 g, more preferably about 1 g.

The concentrated omega-3 fatty acids comprised in formulations of the present invention, is preferably provided in a dose of between 100 mg and 10,000 mg/day, more preferably between 200 mg and 8,000 mg/day, more preferably between 300 mg and 6,000 mg/day, more preferably between 400 mg and 5,000 mg/day, most preferably between 500 mg and 4,000 mg/day.

The formulation may be a single daily dose preparation to give in one dose the above intakes, or may be in convenient divided doses, for example, a daily dose formed of one to four soft gelatin or other dosage forms, each containing 300-1000 mg of omega-3 fatty acids in an appropriate form.

Flavourants may be included, for instance, to make the preparation palatable. Other conventional additives, diluents and excipients may be present. The preparation for ingestion may be in the form of a capsule, a dry powder, a tablet, an oil, an emulsion or any other appropriate form. The capsules may be hard or soft gelatin capsules, agar capsules, or any other appropriate capsule.

Use of the formulations of the invention in the manufacture of a medicament for the treatment or prevention of any disease or disorder, including those mentioned above, is included in the present invention.

The omega-3 fatty acid comprising formulations of the present invention optionally include chemical antioxidants, such as alpha tocopherol, which are administered in pure form or suspended in a vegetable oil, such as soybean oil or corn oil.

The blended fatty acid formulations may then be incorporated into any appropriate dosage form for oral, enteral, parenteral, rectal, vaginal, dermal or other route of administration. Soft or hard gelatin capsules, flavoured oil blends, emulsifiers or other liquid forms, and microencapsulate powders or other dry form vehicles are all appropriate ways of administering the products.

The formulated final drug product containing the omega-3 fatty acid composition formulated according to one or more of the embodiments of the present invention may be administered to a mammal or patient in need thereof in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The combination product may also be contained in a liquid suitable for injection or infusion.

Example pharmaceutical grade finished dosage forms: (a) Soft or hard gelatin capsules each containing 500 mg or 1000 mg of omega-3 fatty acids as a free fatty acid, formulated according to one or more of the embodiments of the present invention; (b) As in (a) but where the omega-3 free fatty acids are replaced with the fatty acids in any other appropriate bioassimilable form such as the ethyl esters; (c) As in (a)-(b) but where the material is in the form of a microencapsulated powder which can be used as a powder or compressed into tablets. Such powders may be prepared by a variety of technologies known to those skilled in the art; (d) As in (a)-(b) but where the formulation is a liquid or emulsion, appropriately flavoured for palatable oral administration; (e) As in (a)-(b) but where the material is formulated into a pharmaceutically acceptable vehicle appropriate for topical application such as a cream or ointment.

The omega-3 formulations of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthan gum.

The term “pharmaceutically acceptable vehicle,” as used herein, includes any of the following: a solution where the first API and optional other ingredients are wholly dissolved in a solubilizer (e.g., a pharmaceutically acceptable solvent or mixture of solvents), wherein the solution remains in clear liquid form at about room temperature; a suspension; an oil; or a semi-solid, wherein the first API and optionally other ingredients are dissolved wholly or partially in a solubilizer (e.g., an emulsion, cream, etc.).

A “pharmaceutical grade finished dosage form” as used herein may be construed as a unit dose form suitable for administration to, for example, human or animal subjects, and having content uniformity acceptable to regulatory authorities. For example, under the USP requirements for content uniformity, a pharmaceutical grade finished dosage form should have an amount of API within the range of 85% to 115% of the desired dosage and an RSD less than or equal to 6.0%. In addition, a pharmaceutical grade finished dosage form must be stable (i.e., have a “shelf life”) for a pharmaceutically acceptable duration of time, preferably at least six months, more preferably at least one year, and most preferably at least two years, when stored at room temperature (about 23° C. to 27° C., preferably about 25° C.) and 60% relative humidity. Typically, stability is determined by physical appearance and/or chemical modification of the ingredients, in accordance with standards well-known in the pharmaceutical arts, including those documented in ICH guidelines.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above. Therefore, the scope of the appended claims should be accorded the broadest interpretation so as to encompass all such modification.

### EXAMPLES

### Example 1

A composition according to the present prevention is prepared by mixing and homogenizing the intermediates Megapex E90D00EE and Megapex D80EE in a ratio of 88:12. Both intermediates were prepared and commercially offered for sale by Chemport Korea. This composition comprises 80.66% EPA, 9.97% DHA, 92.83% total omega-3 fatty acids and 3.95% omega-6 fatty acids (all Area %).

### Example 2

A composition according to the present prevention is prepared by mixing and homogenizing the intermediates Megapex E90D00EE and Megapex D80EE in a ratio of 97:3. Both intermediates were prepared and commercially offered for sale by Chemport Korea. This composition comprises 88.36% EPA, 2.49% DHA, 92.96% total omega-3 fatty acids and 3.72% omega-6 fatty acids (all Area %).

### Example 3

A composition according to the present prevention is prepared by mixing and homogenizing the intermediates Megapex E90D00EE and Megapex D80EE in a ratio of 39:1. Both intermediates were prepared and commercially offered for sale by Chemport Korea. This composition comprises 88.78% EPA, 2.08% DHA, 92.96% total omega-3 fatty acids and 3.70% omega-6 fatty acids (all Area %).

### Example 4

The ethyl ester composition of Example 1 is converted into a free fatty acid composition with essentially the same fatty acid composition according to “Conversion Method EE to FFA” below. This method is indiscriminate with respect to the type, degree of saturation or length of fatty acid if performed for an adequate amount of time under the described conditions.

Conversion Method EE to FFA
1. Fatty Acid Ethyl Ester (FAEE GMP, approx. 3 mmol/g) oil is brought into a closed heated/cooled reaction chamber under nitrogen atmosphere (preferably with pressure control), and heated to 50-60 degree Celcius under stirring.
2. 2M NaOH solution in water is added under firm stirring to ensure phase mixing (est. 2-3×FAEE w/w) and stir until no ethyl ester is presence (est. 2-4 hrs). Test ethyl ester presence at lab scale/in process with TLC (hexanes/EtOAc 9:1) and with EP GC method to confirm reaction completion under GMP.
3. Under cooling (keep mixture below 70 degree Celcius), add 6M HCl in water (est. <1 hr) until slightly acid (˜pH3-4). It may be necessary to control pressure to event excessive foaming. Then halt stirring, give time to let phases separate, and remove water phase from bottom (keep oil protected from oxygen, apply nitrogen atmosphere blanket).
4. Add demineralized water (est. 2-3×FAEE w/w) and wash out NaCl and ethanol from oil under firm stirring (est. ˜1 hr). Halt stirring, give time to let phases separate, and remove water phase from bottom (keep oil protected from oxygen, apply nitrogen atmosphere blanket).
5. Repeat Step 4 several times (˜2×) to remove ethanol and NaCl.
6. Remove water and remaining ethanol [determine in-process control], confirm under GMP with USP residual solvent method (target: ethanol <100 ppm) by applying vacuum 10-50 mbar (with solvent trap) and heat oil (˜80 degree celcius) while stirring firmly until water/ethanol target is met (est. 2-4 hrs)
7. Add anti-oxidants (i.e. alpha-D-tocopherol, USP, target 4 mg/g) and other excipients
8. All reagents and excipients USP grade

### Example 5

The ethyl ester composition of Example 2 is converted into a free fatty acid composition with essentially the same fatty acid composition according to “Conversion Method EE to FFA” above. This method is indiscriminate with respect to the type, degree of saturation or length of fatty acid if performed for an adequate amount of time under the described conditions.

### Example 6

The ethyl ester composition of Example 3 is converted into a free fatty acid composition with essentially the same fatty acid composition according to “Conversion Method EE to FFA” above. This method is indiscriminate with respect to the type, degree of saturation or length of fatty acid if performed for an adequate amount of time under the described conditions.

### Example 7

The composition of Example 4 is formulated into a soft gelatin capsule. Prior to encapsulation, 4 g alpha-D-tocopherol, 4 g BHA, 4 g THBP, 10 g BAC, 10 g HSA and 10 g glycerol-mono-oleate is added to one kg of the oil composition of Example 4 followed by thorough homogenization. Subsequently, the formulated oil is encapsulated into soft gelatin capsules with printed logo according to general methods typically used by Accucaps in Canada for fish oils, except that the gelatin contain approximately 20 mg alpha-tocopherol-polyethylene-glycol-succinate per gram of gelatin upon drying. The fill mass of the oil is 1.29 gram/capsule, providing a dose of approximately 1000 mg eicosapentaenoic acid per capsule. Finally, the capsules are bottled in HDPE bottles with induction seal and child resistant cap.

### Example 8

The composition of Example 6 is formulated into a soft gelatin capsule. Prior to encapsulation, 4 g alpha-D-tocopherol, 4 g BHT, and 10 g glycerol-mono-oleate is added to one kg of the oil composition of Example 5 followed by thorough homogenization. Subsequently, the formulated oil is encapsulated into soft gelatin capsules with printed logo according to general methods typically used by Accucaps in Canada for fish oils. The fill mass of the oil is 1.16 gram/capsule, providing a dose of approximately 1000 mg eicosapentaenoic acid per capsule. Finally, the capsules are bottled in HDPE bottles with induction seal and child resistant cap.

### Example 9

The composition of Example 3 is formulated into a soft gelatin capsule. Prior to encapsulation, 4 g alpha-D-tocopherol, 4 g BHT, and 10 g glycerol-mono-oleate is added to one kg of the oil composition of Example 3 followed by thorough homogenization. Subsequently, the formulated oil is encapsulated into soft gelatin capsules with printed logo according to general methods typically used by Accucaps in Canada for fish oils. The fill mass of the oil is 1.29 gram/capsule, providing a dose of approximately 1000 mg ethyl eicosapentaenoate per capsule. Finally, the capsules are bottled in HDPE bottles with induction seal and child resistant cap.

### Example 10

The composition of Example 6 is formulated into a soft gelatin capsule. Prior to encapsulation, 4 g alpha-D-tocopherol, 4 g BHT, 4 g TBHQ, and 10 g glycerol-mono-oleate is added to one kg of the oil composition of Example 5 followed by thorough homogenization. Subsequently, the formulated oil is encapsulated into soft gelatin capsules with printed logo according to general methods typically used by Accucaps in Canada for fish oils. The fill mass of the oil is 1.16 gram/capsule, providing a dose of approximately 1000 mg eicosapentaenoic acid per capsule. Finally, the capsules are bottled in HDPE bottles with induction seal and child resistant cap.

### Example 11

The composition of Example 6 is formulated into a soft gelatin capsule. Prior to encapsulation, 4 g alpha-D-tocopherol, 4 g BHT, 4 g TBHQ, 10 g TPGS, and 10 g sorbitan mono-oleate is added to one kg of the oil composition of Example 5 followed by thorough homogenization. Subsequently, the formulated oil is encapsulated into soft gelatin capsules with printed logo according to general methods typically used by Accucaps in Canada for fish oils. The fill mass of the oil is 1.16 gram/capsule, providing a dose of approximately 1000 mg eicosapentaenoic acid per capsule. Finally, the capsules are bottled in HDPE bottles with induction seal and child resistant cap.

### Example 12

The composition of Example 3 is formulated into a soft gelatin capsule. Prior to encapsulation, 4 g alpha-D-tocopherol, 4 g BHT, 4 g TBHQ, and 10 g glycerol-mono-oleate is added to one kg of the oil composition of Example 3 followed by thorough homogenization. Subsequently, the formulated oil is encapsulated into soft gelatin capsules with printed logo according to general methods typically used by Accucaps in Canada for fish oils. The fill mass of the oil is 1.29 gram/capsule, providing a dose of approximately 1000 mg ethyl eicosapentaenoate per capsule. Finally, the capsules are bottled in HDPE bottles with induction seal and child resistant cap.

### Example 13

A patient is diagnosed with severe hypertriglyceridemia (TG>500 mg/dL). Thereupon, the patient is initiated on daily treatment with one of the encapsulated compositions according to Examples 7, 8, 9, 10, 11 or 12. Four capsules per day are administered to this patient, resulting in a daily dose of 4000 mg eicosapentaenoic acid (Examples 7, 9 or 12) or ethyl eicosapentaenoate (Examples 8, 10 or 11).

### Example 14

A patient is treated as per Example 13. The treatment results in significant reduction of TG as well as non-HDL- and VLDL-cholesterol levels while the LDL-cholesterol level changes insignificantly.

### Example 15

A patient already undergoing treatment with a statin is diagnosed with high triglycerides (TG between 200 and 500 mg/dL). Thereupon, the patient is initiated on daily treatment with one of the encapsulated compositions according to Examples 7, 8, 9, 10, 11 or 12. Four capsules per day are administered to this patient, resulting in a daily dose of 4000 mg eicosapentaenoic acid (Examples 8, 10, or 11) or ethyl eicosapentaenoate (Examples 7, 9 or 12).

### Example 16

A patient is treated as per Example 15. The treatment results in significant reduction of TG as well as non-HDL-, VLDL- and LDL-cholesterol levels.

### Example 17

A patient is diagnosed with mixed dyslipidemia (TG between 200 and 500 mg/dL and LDL-cholesterol above 190 mg/dL). Thereupon, the patient is initiated on concomitant daily treatment with a statin and one of the encapsulated compositions according to Examples 7, 8 or 9 is. Four capsules per day are administered to this patient, resulting in a daily dose of 4000 mg eicosapentaenoic acid (Examples 8, 10, or 11) or ethyl eicosapentaenoate (Examples 7, 9 or 12).

### Example 18

A patient is treated as per Example 17. The treatment results in significant reduction of TG as well as non-HDL-, VLDL- and LDL-cholesterol levels.

### Example 19

A patient is diagnosed to be at high risk for a cardiovascular event according to the NCEP guidelines and has TG levels above 150 mg/dL. Thereupon, the patient is initiated on daily treatment with one of the encapsulated compositions according to Examples 7, 8 or 9 is. Four capsules per day are administered to this patient, resulting in a daily dose of 4000 mg eicosapentaenoic acid (Examples 7 or 8) or ethyl eicosapentaenoate (Example 9).

### Example 20

A patient is treated as per Example 19. The treatment results in significant reduction of TG as well as non-HDL-, VLDL- and LDL-cholesterol levels.

### Example 21

A patient diagnosed as per Example 13, 15, 17, or 19 is treated with 3 capsules per day (instead of 4) of one of the encapsulated compositions according to Examples 7, 8 or 9. The treatment results in significant reduction of TG as well as non-HDL- and VLDL-cholesterol levels.

### Example 22

A patient diagnosed as per Example 13, 15, 17, or 19 is treated with 2 capsules per day (instead of 3 or 4) of one of the encapsulated compositions according to Examples 7, 8 or 9. The treatment results in significant reduction of TG as well as non-HDL- and VLDL-cholesterol levels.

### Example 23

A patient is treated as per Example 21 or 22. The treatment results in significant reduction of TG as well as non-HDL-, VLDL- and LDL-cholesterol levels.

### Description of Exemplary Embodiments

1. A fatty acid formulation comprising an adjuvant and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
2. A fatty acid formulation comprising at least 30% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
3. A fatty acid formulation comprising at least 65% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
4. A fatty acid formulation comprising at least 80% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
5. A fatty acid formulation comprising at least 85% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
6. A fatty acid formulation comprising at least 90% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
7. A fatty acid formulation comprising between 80% and 95% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
8. A fatty acid formulation comprising between 85% and 95% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
9. A fatty acid formulation comprising between 90% and 95% omega-3 fatty acids, an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
10. A fatty acid formulation comprising between 80% and 95% EPA, at least 2% DHA, less than 4.5% arachidonic acid (C22:4n6), an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
11. A fatty acid formulation comprising between 85% and 95% EPA, at least 2% DHA, less than 4% arachidonic acid (C22:4n6), an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
12. A fatty acid formulation comprising between 90% and 95% EPA, at least 2% DHA, less than 4% arachidonic acid (C22:4n6), an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
13. A fatty acid formulation comprising between 90% and 95% EPA, at least 1% DHA, less than 3% arachidonic acid (C22:4n6), an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
14. A fatty acid formulation comprising between 90% and 95% EPA, at least 1% DHA, less than 2.25% arachidonic acid (C22:4n6), an adjuvant, and at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammonium chloride (BAC)
   h. hexylsalicylic acid (HSA)
15. A fatty acid formulation according to one of the preferred embodiments 1 through 14, in which at least one of the following absorption enhancers are present in the following amounts:
   a. propyl gallate up to 5 mg/g
   b. butylated hydroxyanisole (BHA) up to 5 mg/g
   c. butylated hydroxytoluene (BHT) up to 5 mg/g
   d. tertiary butylhydroquinone (TBHQ) up to 5 mg/g
   e. 2,4,5-trihydroxybutyrophenone (THBP) up to 5 mg/g
   f. alpha-tocopheryl polyethylene glycol succinate (TPGS, tocophersolan) up to 20 mg/g
   g. hexadecyldimethylbenzylammonium chloride (BAC) up to 20 mg/g
   h. hexylsalicylic acid (HSA) up to 20 mg/g
16. A fatty acid formulation according to one of the preferred embodiments 1 through 14, in which at least one of the following absorption enhancers are present in the following amounts:
   a. propyl gallate up to 2.5 mg/g
   b. butylated hydroxyanisole (BHA) up to 2.5 mg/g
   c. butylated hydroxytoluene (BHT) up to 2.5 mg/g
   d. tertiary butylhydroquinone (TBHQ) up to 2.5 mg/g
   e. 2,4,5-trihydroxybutyrophenone (THBP) up to 2.5 mg/g
   f. alpha-tocopheryl polyethylene glycol succinate (TPGS, tocophersolan) up to 10 mg/g
   g. hexadecyldimethylbenzylammonium chloride (BAC) up to 10 mg/g
   h. hexylsalicylic acid (HSA) up to 10 mg/g
17. A fatty acid formulation according to one of the preferred embodiments 1 through 14, in which at least one of the following absorption enhancers are present in the following amounts:
   a. propyl gallate up to 1 mg/g
   b. butylated hydroxyanisole (BHA) up to 1 mg/g
   c. butylated hydroxytoluene (BHT) up to 1 mg/g
   d. tertiary butylhydroquinone (TBHQ) up to 1 mg/g
   e. 2,4,5-trihydroxybutyrophenone (THBP) up to 1 mg/g
   f. alpha-tocopheryl polyethylene glycol succinate (TPGS, tocophersolan) up to 5 mg/g
   g. hexadecyldimethylbenzylammonium chloride (BAC) up to 5 mg/g
   h. hexylsalicylic acid (HSA) up to 5 mg/g
18. A fatty acid formulation according to one of the preferred embodiments 1 through 14, in which at least one of the following absorption enhancers are present in the following amounts:
   a. propyl gallate up to 0.5 mg/g
   b. butylated hydroxyanisole (BHA) up to 0.5 mg/g
   c. butylated hydroxytoluene (BHT) up to 0.5 mg/g
   d. tertiary butylhydroquinone (TBHQ) up to 0.5 mg/g
   e. 2,4,5-trihydroxybutyrophenone (THBP) up to 0.5 mg/g
   f. alpha-tocopheryl polyethylene glycol succinate (TPGS, tocophersolan) up to 2.5 mg/g
   g. hexadecyldimethylbenzylammonium chloride (BAC) up to 2.5 mg/g
   h. hexylsalicylic acid (HSA) up to 2.5 mg/g
19. A fatty acid formulation according to one of the preferred embodiments 1 through 14, in which at least one of the following absorption enhancers are present in the following amounts:
   a. propyl gallate up to 0.2 mg/g
   b. butylated hydroxyanisole (BHA) up to 0.2 mg/g
   c. butylated hydroxytoluene (BHT) up to 0.2 mg/g
   d. tertiary butylhydroquinone (TBHQ) up to 0.2 mg/g
   e. 2,4,5-trihydroxybutyrophenone (THBP) up to 0.2 mg/g
   f. alpha-tocopheryl polyethylene glycol succinate (TPGS, tocophersolan) up to 1 mg/g
   g. hexadecyldimethylbenzylammonium chloride (BAC) up to 1 mg/g
   h. hexylsalicylic acid (HSA) up to 1 mg/g
20. A fatty acid formulation according to one of the preferred embodiments 1 through 19, in which the adjuvant is chosen from:
   a. Glyceryl mono-oleate
   b. Sorbitan mono-oleate
   c. Polyglyceryl-3-oleate
21. A fatty acid formulation according to one of the preferred embodiments 1 through 20, in which the fatty acids are present as ethyl esters.
22. A fatty acid formulation according to one of the preferred embodiments 1 through 20, in which the fatty acids are present in the form of triglycerides
23. A fatty acid formulation according to one of the preferred embodiments 1 through 20, in which at least 60% the fatty acids are present in the form of triglycerides
24. A fatty acid formulation according to one of the preferred embodiments 1 through 20, in which at least 70% the fatty acids are present in the form of triglycerides
25. A fatty acid formulation according to one of the preferred embodiments 1 through 20, in which more than 90% the fatty acids are present in the form of triglycerides
26. A fatty acid formulation according to one of the preferred embodiments 1 through 20, in which the fatty acids are present in the form of di-glycerides
27. A fatty acid formulation according to one of the preferred embodiments 1 through 20, in which the fatty acids are present in the form of mono-glycerides
28. A fatty acid formulation according to one of the preferred embodiments 1 through 20, in which the fatty acids are present as free fatty acids.
29. A fatty acid formulation according to one of the preferred embodiments 1 through 20, in which the fatty acids are present in the form of salts, esters of any type, amides, or phospholipids.
30. A fatty acid formulation according to one of the preferred embodiments 1 through 29, in which the fatty acids are present in an amount of at least 90%
31. A fatty acid formulation according to one of the preferred embodiments 1 through 29, in which the fatty acids are present in an amount of at least 95%
32. A fatty acid formulation according to one of the preferred embodiments 1 through 29, in which the fatty acids are present in an amount of at least 96%
33. A fatty acid formulation according to one of the preferred embodiments 1 through 29, in which the fatty acids are present in an amount of at least 97%
34. A pharmaceutical composition comprising a fatty acid formulation according to one of the preferred embodiments 1 through 33 contained in a soft or hard gelatin capsule shell.
35. A pharmaceutical composition according to one of the preferred embodiment 34, contained in a soft or hard gelatin capsule shell in which the gelatin comprises at least one of the following absorption enhancers:
   a. propyl gallate
   b. butylated hydroxyanisole (BHA)
   c. butylated hydroxytoluene (BHT)
   d. tertiary butylhydroquinone (TBHQ)
   e. 2,4,5-trihydroxybutyrophenone (THBP)
   f. alpha-tocopheryl polyethylene glycol succinate (TPGS, tocophersolan)
   g. hexadecyldimethylbenzylammoniurn chloride (BAC)
   h. hexylsalicylic acid (HSA)
36. A pharmaceutical composition according to preferred embodiment 35, in which the dried gelatin capsule shell comprises at least one of the following absorption enhancers are present in the following amounts:
   a. propyl gallate up to 10 mg/g
   b. butylated hydroxyanisole (BHA) up to 10 mg/g
   c. butylated hydroxytoluene (BHT) up to 10 mg/g
   d. tertiary butylhydroquinone (TBHQ) up to 10 mg/g
   e. 2,4,5-trihydroxybutyrophenone (THBP) up to 10 mg/g
   f. alpha-tocopheryl polyethylene glycol succinate (TPGS, tocophersolan) up to 50 mg/g
   g. hexadecyldimethylbenzylammonium chloride (BAC) up to 50 mg/g
   h. hexylsalicylic acid (HSA) up to 50 mg/g
37. A pharmaceutical composition according to preferred embodiment 35, in which the dried gelatin capsule shell comprises at least one of the following absorption enhancers are present in the following amounts:
   a. propyl gallate up to 5 mg/g
   b. butylated hydroxyanisole (BHA) up to 5 mg/g
   c. butylated hydroxytoluene (BHT) up to 5 mg/g
   d. tertiary butylhydroquinone (TBHQ) up to 5 mg/g
   e. 2,4,5-trihydroxybutyrophenone (THBP) up to 5 mg/g
   f. alpha-tocopheryl polyethylene glycol succinate (TPGS, tocophersolan) up to 20 mg/g
   g. hexadecyldimethylbenzylammonium chloride (BAC) up to 20 mg/g
   h. hexylsalicylic acid (HSA) up to 20 mg/g
38. A pharmaceutical composition according to preferred embodiment 35, in which the dried gelatin capsule shell comprises at least one of the following absorption enhancers are present in the following amounts:
   a. propyl gallate up to 2.5 mg/g
   b. butylated hydroxyanisole (BHA) up to 2.5 mg/g
   c. butylated hydroxytoluene (BHT) up to 2.5 mg/g
   d. tertiary butylhydroquinone (TBHQ) up to 2.5 mg/g
   e. 2,4,5-trihydroxybutyrophenone (THBP) up to 2.5 mg/g
   f. alpha-tocopheryl polyethylene glycol succinate (TPGS, tocophersolan) up to 10 mg/g
   g. hexadecyldimethylbenzylammonium chloride (BAC) up to 10 mg/g
   h. hexylsalicylic acid (HSA) up to 10 mg/g
39. A pharmaceutical composition according to preferred embodiment 35, in which the dried gelatin capsule shell comprises at least one of the following absorption enhancers are present in the following amounts:
   a. propyl gallate up to 1 mg/g
   b. butylated hydroxyanisole (BHA) up to 1 mg/g
   c. butylated hydroxytoluene (BHT) up to 1 mg/g
   d. tertiary butylhydroquinone (TBHQ) up to 1 mg/g
   e. 2,4,5-trihydroxybutyrophenone (THBP) up to 1 mg/g
   f. alpha-tocopheryl polyethylene glycol succinate (TPGS, tocophersolan) up to 5 mg/g
   g. hexadecyldimethylbenzylammonium chloride (BAC) up to 5 mg/g
   h. hexylsalicylic acid (HSA) up to 5 mg/g
40. A pharmaceutical composition according to preferred embodiment 35, in which the dried gelatin capsule shell comprises at least one of the following absorption enhancers are present in the following amounts:
   a. propyl gallate up to 0.5 mg/g
   b. butylated hydroxyanisole (BHA) up to 0.5 mg/g
   c. butylated hydroxytoluene (BHT) up to 0.5 mg/g
   d. tertiary butylhydroquinone (TBHQ) up to 0.5 mg/g
   e. 2,4,5-trihydroxybutyrophenone (THBP) up to 0.5 mg/g
   f. alpha-tocopheryl polyethylene glycol succinate (TPGS, tocophersolan) up to 2.5 mg/g
   g. hexadecyldimethylbenzylammonium chloride (BAC) up to 2.5 mg/g
   h. hexylsalicylic acid (HSA) up to 2.5 mg/g
41. A pharmaceutical composition according to preferred embodiment 35, in which the dried gelatin capsule shell comprises at least one of the following absorption enhancers are present in the following amounts:
   a. propyl gallate up to 0.2 mg/g
   b. butylated hydroxyanisole (BHA) up to 0.2 mg/g
   c. butylated hydroxytoluene (BHT) up to 0.2 mg/g
   d. tertiary butylhydroquinone (TBHQ) up to 0.2 mg/g
   e. 2,4,5-trihydroxybutyrophenone (THBP) up to 0.2 mg/g
   f. alpha-tocopheryl polyethylene glycol succinate (TPGS, tocophersolan) up to 1 mg/g
   g. hexadecyldimethylbenzylammonium chloride (BAC) up to 1 mg/g
   h. hexylsalicylic acid (HSA) up to 1 mg/g
42. A fatty acid composition according to one of the preferred embodiments 1 through 33, also comprising a suitable anti-oxidant in a concentration sufficient to protect the fatty acids of the composition from oxidation.
43. A pharmaceutical composition according to one of the preferred embodiments 34 through 41, also comprising a suitable anti-oxidant in a concentration sufficient to protect the fatty acids of the composition from oxidation.
44. A pharmaceutically suitable dosage form comprising one of the formulations or compositions according to preferred embodiments 1 through 43, in which the amount of eicosapentaenoic acid is present in an amount between 250 and 10,000 mg.
45. A pharmaceutically suitable dosage form comprising one of the formulations or compositions according to preferred embodiments 1 through 43, in which the amount of eicosapentaenoic acid is present in an amount between 250 and 1,250 mg.
46. A pharmaceutically suitable dosage form comprising one of the formulations or compositions according to preferred embodiments 1 through 43, in which the amount of eicosapentaenoic acid is present in an amount between 500 and 1,100 mg.
47. A method of administration or treatment to a subject of a formulation according to one of the preferred embodiments 44 through 46 at a daily dose between 100 and 10,000 mg.
48. A method of administration or treatment to a subject of a formulation according to one of the preferred embodiments 44 through 46 at a daily dose between 500 and 5,000 mg.
49. A method of administration or treatment to a subject of a formulation according to one of the preferred embodiments 44 through 46 at a daily dose of approximately 4 gram
50. A method of administration or treatment under low fat meal conditions according to one of the preferred embodiments 47 through 49 resulting in a Tmax of 8 hours or less
51. A method of administration or treatment under low fat meal conditions according to one of the preferred embodiments 47 through 49 resulting in a Tmax of 6 hours or less
52. A method of administration or treatment under low fat meal conditions according to one of the preferred embodiments 47 through 49 resulting in a Tmax of 5 hours or less
53. A method of administration or treatment under low fat meal conditions according to one of the preferred embodiments 47 through 49 resulting in a Tmax of 4 hours or less
54. A method of administration or treatment under fasting conditions according to one of the preferred embodiments 47 through 49 resulting in a Tmax of 8 hours or less
55. A method of administration or treatment under fasting conditions according to one of the preferred embodiments 47 through 49 resulting in a Tmax of 6 hours or less
56. A method of administration or treatment under fasting conditions according to one of the preferred embodiments 47 through 49 resulting in a Tmax of 5 hours or less
57. A method of administration or treatment under fasting conditions according to one of the preferred embodiments 47 through 49 resulting in a Tmax of 4 hours or less
58. A method of administration or treatment under low fat meal conditions according to one of the preferred embodiments 47 through 49 resulting in an EPA+DHA Tmax less than the EPA+DHA Tmax for Lovaza under the same administration conditions
59. A method of administration or treatment under fasting conditions according to one of the preferred embodiments 47 through 49 resulting in an EPA+DHA Tmax less than the EPA+DHA Tmax for Lovaza under the same administration conditions
60. A method of administration or treatment under low fat meal conditions according to one of the preferred embodiments 47 through 49 resulting in an EPA+DHA Tmax less than the EPA+DHA Tmax for AMR101 under the same administration conditions
61. A method of administration or treatment under fasting conditions according to one of the preferred embodiments 47 through 49 resulting in an EPA+DHA Tmax less than the EPA+DHA Tmax for AMR101 under the same administration conditions
62. A method of administration or treatment under low fat meal conditions according to one of the preferred embodiments 47 through 49 resulting in an Total Omega-3 FA Tmax less than the Total Omega-3 FA Tmax for Lovaza under the same administration conditions
63. A method of administration or treatment under fasting conditions according to one of the preferred embodiments 47 through 49 resulting in an Total Omega-3 FA Tmax less than the Total Omega-3 FA Tmax for Lovaza under the same administration conditions
64. A method of administration or treatment under low fat meal conditions according to one of the preferred embodiments 47 through 49 resulting in an Total Omega-3 FA Tmax less than the Total Omega-3 FA Tmax for AMR101 under the same administration conditions
65. A method of administration or treatment under fasting conditions according to one of the preferred embodiments 47 through 49 resulting in an Total Omega-3 FA Tmax less than the Total Omega-3 FA Tmax for AMR101 under the same administration conditions
66. A method of administration or treatment under high fat meal conditions according to one of the preferred embodiments 47 through 49 resulting in an EPA+DHA Cmax or Total Omega-3 FA Cmax of at least 110% of the EPA+DHA Cmax or Total Omega-3 FA Cmax for Lovaza under the same administration conditions
67. A method of administration or treatment under high fat meal conditions according to one of the preferred embodiments 47 through 49 resulting in an EPA+DHA Cmax or Total Omega-3 FA Cmax of at least 120% of the EPA+DHA Cmax or Total Omega-3 FA Cmax for Lovaza under the same administration conditions
68. A method of administration or treatment under high fat meal conditions according to one of the preferred embodiments 47 through 49 resulting in an EPA+DHA Cmax or Total Omega-3 FA Cmax of at least 130% of the EPA+DHA Cmax or Total Omega-3 FA Cmax for Lovaza under the same administration conditions
69. A method of administration or treatment under high fat meal conditions according to one of the preferred embodiments 47 through 49 resulting in an EPA+DHA Cmax or Total Omega-3 FA Cmax of at least 140% of the EPA+DHA Cmax or Total Omega-3 FA Cmax for Lovaza under the same administration conditions
70. A method of administration or treatment under high fat meal conditions according to one of the preferred embodiments 47 through 49 resulting in an EPA+DHA Cmax or Total Omega-3 FA Cmax of at least 150% of the EPA+DHA Cmax or Total Omega-3 FA Cmax for Lovaza under the same administration conditions
71. A method of administration or treatment under low fat meal conditions according to one of the preferred embodiments 47 through 49 resulting in an EPA+DHA Cmax or Total Omega-3 FA Cmax of at least 150% of the EPA+DHA Cmax or Total Omega-3 FA Cmax for Lovaza under the same administration conditions
72. A method of administration or treatment under low fat meal conditions according to one of the preferred embodiments 47 through 49 resulting in an EPA+DHA Cmax or Total Omega-3 FA Cmax of at least 200% of the EPA+DHA Cmax or Total Omega-3 FA Cmax for Lovaza under the same administration conditions
73. A method of administration or treatment under low fat meal conditions according to one of the preferred embodiments 47 through 49 resulting in an EPA+DHA Cmax or Total Omega-3 FA Cmax of at least 300% of the EPA+DHA Cmax or Total Omega-3 FA Cmax for Lovaza under the same administration conditions
74. A method of administration or treatment under low fat meal conditions according to one of the preferred embodiments 47 through 49 resulting in an EPA+DHA Cmax or Total Omega-3 FA Cmax of at least 400% of the EPA+DHA Cmax or Total Omega-3 FA Cmax for Lovaza under the same administration conditions
75. A method of administration or treatment under low fat meal conditions according to one of the preferred embodiments 47 through 49 resulting in an EPA+DHA Cmax or Total Omega-3 FA Cmax of at least 500% of the EPA+DHA Cmax or Total Omega-3 FA Cmax for Lovaza under the same administration conditions
76. A method of administration or treatment under low fat meal conditions according to one of the preferred embodiments 47 through 49 resulting in an EPA+DHA Cmax or Total Omega-3 FA Cmax of at least 600% of the EPA+DHA Cmax or Total Omega-3 FA Cmax for Lovaza under the same administration conditions
77. A method of administration or treatment under fasting conditions according to one of the preferred embodiments 47 through 49 resulting in an EPA+DHA Cmax or Total Omega-3 FA Cmax of at least 150% of the EPA+DHA Cmax or Total Omega-3 FA Cmax for Lovaza under the same administration conditions
78. A method of administration or treatment under fasting conditions according to one of the preferred embodiments 47 through 49 resulting in an EPA+DHA Cmax or Total Omega-3 FA Cmax of at least 200% of the EPA+DHA Cmax or Total Omega-3 FA Cmax for Lovaza under the same administration conditions
79. A method of administration or treatment under fasting conditions according to one of the preferred embodiments 47 through 49 resulting in an EPA+DHA Cmax or Total Omega-3 FA Cmax of at least 300% of the EPA+DHA Cmax or Total Omega-3 FA Cmax for Lovaza under the same administration conditions
80. A method of administration or treatment under fasting conditions according to one of the preferred embodiments 47 through 49 resulting in an EPA+DHA Cmax or Total Omega-3 FA Cmax of at least 400% of the EPA+DHA Cmax or Total Omega-3 FA Cmax for Lovaza under the same administration conditions
81. A method of administration or treatment under fasting conditions according to one of the preferred embodiments 47 through 49 resulting in an EPA+DHA Cmax or Total Omega-3 FA Cmax of at least 500% of the EPA+DHA Cmax or Total Omega-3 FA Cmax for Lovaza under the same administration conditions
82. A method of administration or treatment under fasting conditions according to one of the preferred embodiments 47 through 49 resulting in an EPA+DHA Cmax or Total Omega-3 FA Cmax of at least 600% of the EPA+DHA Cmax or Total Omega-3 FA Cmax for Lovaza under the same administration conditions
83. A method of administration or treatment under fasting conditions according to one of the preferred embodiments 47 through 49 resulting in an EPA+DHA Cmax or Total Omega-3 FA Cmax of at least 700% of the EPA+DHA Cmax or Total Omega-3 FA Cmax for Lovaza under the same administration conditions
84. A method of administration or treatment under high fat meal conditions according to one of the preferred embodiments 47 through 49 resulting in an EPA+DHA AUC or Total Omega-3 FA AUC of at least 110% of the EPA+DHA AUC or Total Omega-3 FA AUC for Lovaza under the same administration conditions
85. A method of administration or treatment under high fat meal conditions according to one of the preferred embodiments 47 through 49 resulting in an EPA+DHA AUC or Total Omega-3 FA AUC of at least 120% of the EPA+DHA AUC or Total Omega-3 FA AUC for Lovaza under the same administration conditions
86. A method of administration or treatment under high fat meal conditions according to one of the preferred embodiments 47 through 49 resulting in an EPA+DHA AUC or Total Omega-3 FA AUC of at least 130% of the EPA+DHA AUC or Total Omega-3 FA AUC for Lovaza under the same administration conditions
87. A method of administration or treatment under high fat meal conditions according to one of the preferred embodiments 47 through 49 resulting in an EPA+DHA AUC or Total Omega-3 FA AUC of at least 140% of the EPA+DHA AUC or Total Omega-3 FA AUC for Lovaza under the same administration conditions
88. A method of administration or treatment under high fat meal conditions according to one of the preferred embodiments 47 through 49 resulting in an EPA+DHA AUC or Total Omega-3 FA AUC of at least 150% of the EPA+DHA AUC or Total Omega-3 FA AUC for Lovaza under the same administration conditions
89. A method of administration or treatment under low fat meal conditions according to one of the preferred embodiments 47 through 49 resulting in an EPA+DHA AUC or Total Omega-3 FA AUC of at least 150% of the EPA+DHA AUC or Total Omega-3 FA AUC for Lovaza under the same administration conditions
90. A method of administration or treatment under low fat meal conditions according to one of the preferred embodiments 47 through 49 resulting in an EPA+DHA AUC or Total Omega-3 FA AUC of at least 200% of the EPA+DHA AUC or Total Omega-3 FA AUC for Lovaza under the same administration conditions
91. A method of administration or treatment under low fat meal conditions according to one of the preferred embodiments 47 through 49 resulting in an EPA+DHA AUC or Total Omega-3 FA AUC of at least 300% of the EPA+DHA AUC or Total Omega-3 FA AUC for Lovaza under the same administration conditions
92. A method of administration or treatment under low fat meal conditions according to one of the preferred embodiments 47 through 49 resulting in an EPA+DHA AUC or Total Omega-3 FA AUC of at least 400% of the EPA+DHA AUC or Total Omega-3 FA AUC for Lovaza under the same administration conditions
93. A method of administration or treatment under low fat meal conditions according to one of the preferred embodiments 47 through 49 resulting in an EPA+DHA AUC or Total Omega-3 FA AUC of at least 500% of the EPA+DHA AUC or Total Omega-3 FA AUC for Lovaza under the same administration conditions
94. A method of administration or treatment under low fat meal conditions according to one of the preferred embodiments 47 through 49 resulting in an EPA+DHA AUC or Total Omega-3 FA AUC of at least 600% of the EPA+DHA AUC or Total Omega-3 FA AUC for Lovaza under the same administration conditions
95. A method of administration or treatment under fasting conditions according to one of the preferred embodiments 47 through 49 resulting in an EPA+DHA AUC or Total Omega-3 FA AUC of at least 150% of the EPA+DHA AUC or Total Omega-3 FA AUC for Lovaza under the same administration conditions
96. A method of administration or treatment under fasting conditions according to one of the preferred embodiments 47 through 49 resulting in an EPA+DHA AUC or Total Omega-3 FA AUC of at least 200% of the EPA+DHA AUC or Total Omega-3 FA AUC for Lovaza under the same administration conditions
97. A method of administration or treatment under fasting conditions according to one of the preferred embodiments 47 through 49 resulting in an EPA+DHA AUC or Total Omega-3 FA AUC of at least 300% of the EPA+DHA AUC or Total Omega-3 FA AUC for Lovaza under the same administration conditions
98. A method of administration or treatment under fasting conditions according to one of the preferred embodiments 47 through 49 resulting in an EPA+DHA AUC or Total Omega-3 FA AUC of at least 400% of the EPA+DHA AUC or Total Omega-3 FA AUC for Lovaza under the same administration conditions
99. A method of administration or treatment under fasting conditions according to one of the preferred embodiments 47 through 49 resulting in an EPA+DHA AUC or Total Omega-3 FA AUC of at least 500% of the EPA+DHA AUC or Total Omega-3 FA AUC for Lovaza under the same administration conditions
100. A method of administration or treatment under fasting conditions according to one of the preferred embodiments 47 through 49 resulting in an EPA+DHA AUC or Total Omega-3 FA AUC of at least 600% of the EPA+DHA AUC or Total Omega-3 FA AUC for Lovaza under the same administration conditions
101. A method of administration or treatment under fasting conditions according to one of the preferred embodiments 47 through 49 resulting in an EPA+DHA AUC or Total Omega-3 FA AUC of at least 700% of the EPA+DHA AUC or Total Omega-3 FA AUC for Lovaza under the same administration conditions
102. A method of administration or treatment under high fat meal conditions according to one of the preferred embodiments 47 through 49 resulting in an EPA+DHA Cmax or Total Omega-3 FA Cmax of at least 110% of the EPA+DHA Cmax or Total Omega-3 FA Cmax for AMR101 under the same administration conditions
103. A method of administration or treatment under high fat meal conditions according to one of the preferred embodiments 47 through 49 resulting in an EPA+DHA Cmax or Total Omega-3 FA Cmax of at least 120% of the EPA+DHA Cmax or Total Omega-3 FA Cmax for AMR101 under the same administration conditions
104. A method of administration or treatment under high fat meal conditions according to one of the preferred embodiments 47 through 49 resulting in an EPA+DHA Cmax or Total Omega-3 FA Cmax of at least 130% of the EPA+DHA Cmax or Total Omega-3 FA Cmax for AMR101 under the same administration conditions
105. A method of administration or treatment under high fat meal conditions according to one of the preferred embodiments 47 through 49 resulting in an EPA+DHA Cmax or Total Omega-3 FA Cmax of at least 140% of the EPA+DHA Cmax or Total Omega-3 FA Cmax for AMR101 under the same administration conditions
106. A method of administration or treatment under high fat meal conditions according to one of the preferred embodiments 47 through 49 resulting in an EPA+DHA Cmax or Total Omega-3 FA Cmax of at least 150% of the EPA+DHA Cmax or Total Omega-3 FA Cmax for AMR101 under the same administration conditions
107. A method of administration or treatment under low fat meal conditions according to one of the preferred embodiments 47 through 49 resulting in an EPA+DHA Cmax or Total Omega-3 FA Cmax of at least 150% of the EPA+DHA Cmax or Total Omega-3 FA Cmax for AMR101 under the same administration conditions
108. A method of administration or treatment under low fat meal conditions according to one of the preferred embodiments 47 through 49 resulting in an EPA+DHA Cmax or Total Omega-3 FA Cmax of at least 200% of the EPA+DHA Cmax or Total Omega-3 FA Cmax for AMR101 under the same administration conditions
109. A method of administration or treatment under low fat meal conditions according to one of the preferred embodiments 47 through 49 resulting in an EPA+DHA Cmax or Total Omega-3 FA Cmax of at least 300% of the EPA+DHA Cmax or Total Omega-3 FA Cmax for AMR101 under the same administration conditions
110. A method of administration or treatment under low fat meal conditions according to one of the preferred embodiments 47 through 49 resulting in an EPA+DHA Cmax or Total Omega-3 FA Cmax of at least 400% of the EPA+DHA Cmax or Total Omega-3 FA Cmax for AMR101 under the same administration conditions
111. A method of administration or treatment under low fat meal conditions according to one of the preferred embodiments 47 through 49 resulting in an EPA+DHA Cmax or Total Omega-3 FA Cmax of at least 500% of the EPA+DHA Cmax or Total Omega-3 FA Cmax for AMR101 under the same administration conditions
112. A method of administration or treatment under low fat meal conditions according to one of the preferred embodiments 47 through 49 resulting in an EPA+DHA Cmax or Total Omega-3 FA Cmax of at least 600% of the EPA+DHA Cmax or Total Omega-3 FA Cmax for AMR101 under the same administration conditions
113. A method of administration or treatment under fasting conditions according to one of the preferred embodiments 47 through 49 resulting in an EPA+DHA Cmax or Total Omega-3 FA Cmax of at least 150% of the EPA+DHA Cmax or Total Omega-3 FA Cmax for AMR101 under the same administration conditions
114. A method of administration or treatment under fasting conditions according to one of the preferred embodiments 47 through 49 resulting in an EPA+DHA Cmax or Total Omega-3 FA Cmax of at least 200% of the EPA+DHA Cmax or Total Omega-3 FA Cmax for AMR101 under the same administration conditions
115. A method of administration or treatment under fasting conditions according to one of the preferred embodiments 47 through 49 resulting in an EPA+DHA Cmax or Total Omega-3 FA Cmax of at least 300% of the EPA+DHA Cmax or Total Omega-3 FA Cmax for AMR101 under the same administration conditions
116. A method of administration or treatment under fasting conditions according to one of the preferred embodiments 47 through 49 resulting in an EPA+DHA Cmax or Total Omega-3 FA Cmax of at least 400% of the EPA+DHA Cmax or Total Omega-3 FA Cmax for AMR101 under the same administration conditions
117. A method of administration or treatment under fasting conditions according to one of the preferred embodiments 47 through 49 resulting in an EPA+DHA Cmax or Total Omega-3 FA Cmax of at least 500% of the EPA+DHA Cmax or Total Omega-3 FA Cmax for AMR101 under the same administration conditions
118. A method of administration or treatment under fasting conditions according to one of the preferred embodiments 47 through 49 resulting in an EPA+DHA Cmax or Total Omega-3 FA Cmax of at least 600% of the EPA+DHA Cmax or Total Omega-3 FA Cmax for AMR101 under the same administration conditions
119. A method of administration or treatment under fasting conditions according to one of the preferred embodiments 47 through 49 resulting in an EPA+DHA Cmax or Total Omega-3 FA Cmax of at least 700% of the EPA+DHA Cmax or Total Omega-3 FA Cmax for AMR101 under the same administration conditions
120. A method of administration or treatment under high fat meal conditions according to one of the preferred embodiments 47 through 49 resulting in an EPA+DHA AUC or Total Omega-3 FA AUC of at least 110% of the EPA+DHA AUC or Total Omega-3 FA AUC for AMR101 under the same administration conditions
121. A method of administration or treatment under high fat meal conditions according to one of the preferred embodiments 47 through 49 resulting in an EPA+DHA AUC or Total Omega-3 FA AUC of at least 120% of the EPA+DHA AUC or Total Omega-3 FA AUC for AMR101 under the same administration conditions
122. A method of administration or treatment under high fat meal conditions according to one of the preferred embodiments 47 through 49 resulting in an EPA+DHA AUC or Total Omega-3 FA AUC of at least 130% of the EPA+DHA AUC or Total Omega-3 FA AUC for AMR101 under the same administration conditions
123. A method of administration or treatment under high fat meal conditions according to one of the preferred embodiments 47 through 49 resulting in an EPA+DHA AUC or Total Omega-3 FA AUC of at least 140% of the EPA+DHA AUC or Total Omega-3 FA AUC for AMR101 under the same administration conditions
124. A method of administration or treatment under high fat meal conditions according to one of the preferred embodiments 47 through 49 resulting in an EPA+DHA AUC or Total Omega-3 FA AUC of at least 150% of the EPA+DHA AUC or Total Omega-3 FA AUC for AMR101 under the same administration conditions
125. A method of administration or treatment under low fat meal conditions according to one of the preferred embodiments 47 through 49 resulting in an EPA+DHA AUC or Total Omega-3 FA AUC of at least 150% of the EPA+DHA AUC or Total Omega-3 FA AUC for AMR101 under the same administration conditions
126. A method of administration or treatment under low fat meal conditions according to one of the preferred embodiments 47 through 49 resulting in an EPA+DHA AUC or Total Omega-3 FA AUC of at least 200% of the EPA+DHA AUC or Total Omega-3 FA AUC for AMR101 under the same administration conditions
127. A method of administration or treatment under low fat meal conditions according to one of the preferred embodiments 47 through 49 resulting in an EPA+DHA AUC or Total Omega-3 FA AUC of at least 300% of the EPA+DHA AUC or Total Omega-3 FA AUC for AMR101 under the same administration conditions
128. A method of administration or treatment under low fat meal conditions according to one of the preferred embodiments 47 through 49 resulting in an EPA+DHA AUC or Total Omega-3 FA AUC of at least 400% of the EPA+DHA AUC or Total Omega-3 FA AUC for AMR101 under the same administration conditions
129. A method of administration or treatment under low fat meal conditions according to one of the preferred embodiments 47 through 49 resulting in an EPA+DHA AUC or Total Omega-3 FA AUC of at least 500% of the EPA+DHA AUC or Total Omega-3 FA AUC for AMR101 under the same administration conditions
130. A method of administration or treatment under low fat meal conditions according to one of the preferred embodiments 47 through 49 resulting in an EPA+DHA AUC or Total Omega-3 FA AUC of at least 600% of the EPA+DHA AUC or Total Omega-3 FA AUC for AMR101 under the same administration conditions
131. A method of administration or treatment under fasting conditions according to one of the preferred embodiments 47 through 49 resulting in an EPA+DHA AUC or Total Omega-3 FA AUC of at least 150% of the EPA+DHA AUC or Total Omega-3 FA AUC for AMR101 under the same administration conditions
132. A method of administration or treatment under fasting conditions according to one of the preferred embodiments 47 through 49 resulting in an EPA+DHA AUC or Total Omega-3 FA AUC of at least 200% of the EPA+DHA AUC or Total Omega-3 FA AUC for AMR101 under the same administration conditions
133. A method of administration or treatment under fasting conditions according to one of the preferred embodiments 47 through 49 resulting in an EPA+DHA AUC or Total Omega-3 FA AUC of at least 300% of the EPA+DHA AUC or Total Omega-3 FA AUC for AMR101 under the same administration conditions
134. A method of administration or treatment under fasting conditions according to one of the preferred embodiments 47 through 49 resulting in an EPA+DHA AUC or Total Omega-3 FA AUC of at least 400% of the EPA+DHA AUC or Total Omega-3 FA AUC for AMR101 under the same administration conditions
135. A method of administration or treatment under fasting conditions according to one of the preferred embodiments 47 through 49 resulting in an EPA+DHA AUC or Total Omega-3 FA AUC of at least 500% of the EPA+DHA AUC or Total Omega-3 FA AUC for AMR101 under the same administration conditions
136. A method of administration or treatment under fasting conditions according to one of the preferred embodiments 47 through 49 resulting in an EPA+DHA AUC or Total Omega-3 FA AUC of at least 600% of the EPA+DHA AUC or Total Omega-3 FA AUC for AMR101 under the same administration conditions
137. A method of administration or treatment under fasting conditions according to one of the preferred embodiments 47 through 49 resulting in an EPA+DHA AUC or Total Omega-3 FA AUC of at least 700% of the EPA+DHA AUC or Total Omega-3 FA AUC for AMR101 under the same administration conditions
138. A method of administration or treatment under high fat meal conditions according to one of the preferred embodiments 47 through 49 resulting in an EPA+DHA Cmax or Total Omega-3 FA Cmax of at least 100% of the EPA+DHA Cmax or Total Omega-3 FA Cmax for Epanova under the same administration conditions
139. A method of administration or treatment under high fat meal conditions according to one of the preferred embodiments 47 through 49 resulting in an EPA+DHA Cmax or Total Omega-3 FA Cmax of at least 105% of the EPA+DHA Cmax or Total Omega-3 FA Cmax for Epanova under the same administration conditions
140. A method of administration or treatment under high fat meal conditions according to one of the preferred embodiments 47 through 49 resulting in an EPA+DHA Cmax or Total Omega-3 FA Cmax of at least 110% of the EPA+DHA Cmax or Total Omega-3 FA Cmax for Epanova under the same administration conditions
141. A method of administration or treatment under high fat meal conditions according to one of the preferred embodiments 47 through 49 resulting in an EPA+DHA Cmax or Total Omega-3 FA Cmax of at least 120% of the EPA+DHA Cmax or Total Omega-3 FA Cmax for Epanova under the same administration conditions
142. A method of administration or treatment under high fat meal conditions according to one of the preferred embodiments 47 through 49 resulting in an EPA+DHA Cmax or Total Omega-3 FA Cmax of at least 130% of the EPA+DHA Cmax or Total Omega-3 FA Cmax for Epanova under the same administration conditions
143. A method of administration or treatment under high fat meal conditions according to one of the preferred embodiments 47 through 49 resulting in an EPA+DHA Cmax or Total Omega-3 FA Cmax of at least 140% of the EPA+DHA Cmax or Total Omega-3 FA Cmax for Epanova under the same administration conditions
144. A method of administration or treatment under low fat meal conditions according to one of the preferred embodiments 47 through 49 resulting in an EPA+DHA Cmax or Total Omega-3 FA Cmax of at least 100% of the EPA+DHA Cmax or Total Omega-3 FA Cmax for Epanova under the same administration conditions
145. A method of administration or treatment under low fat meal conditions according to one of the preferred embodiments 47 through 49 resulting in an EPA+DHA Cmax or Total Omega-3 FA Cmax of at least 105% of the EPA+DHA Cmax or Total Omega-3 FA Cmax for Epanova under the same administration conditions
146. A method of administration or treatment under low fat meal conditions according to one of the preferred embodiments 47 through 49 resulting in an EPA+DHA Cmax or Total Omega-3 FA Cmax of at least 110% of the EPA+DHA Cmax or Total Omega-3 FA Cmax for Epanova under the same administration conditions
147. A method of administration or treatment under low fat meal conditions according to one of the preferred embodiments 47 through 49 resulting in an EPA+DHA Cmax or Total Omega-3 FA Cmax of at least 120% of the EPA+DHA Cmax or Total Omega-3 FA Cmax for Epanova under the same administration conditions
148. A method of administration or treatment under low fat meal conditions according to one of the preferred embodiments 47 through 49 resulting in an EPA+DHA Cmax or Total Omega-3 FA Cmax of at least 130% of the EPA+DHA Cmax or Total Omega-3 FA Cmax for Epanova under the same administration conditions
149. A method of administration or treatment under low fat meal conditions according to one of the preferred embodiments 47 through 49 resulting in an EPA+DHA Cmax or Total Omega-3 FA Cmax of at least 140% of the EPA+DHA Cmax or Total Omega-3 FA Cmax for Epanova under the same administration conditions
150. A method of administration or treatment under fasting conditions according to one of the preferred embodiments 47 through 49 resulting in an EPA+DHA Cmax or Total Omega-3 FA Cmax of at least 100% of the EPA+DHA Cmax or Total Omega-3 FA Cmax for Epanova under the same administration conditions
151. A method of administration or treatment under fasting conditions according to one of the preferred embodiments 47 through 49 resulting in an EPA+DHA Cmax or Total Omega-3 FA Cmax of at least 105% of the EPA+DHA Cmax or Total Omega-3 FA Cmax for Epanova under the same administration conditions
152. A method of administration or treatment under fasting conditions according to one of the preferred embodiments 47 through 49 resulting in an EPA+DHA Cmax or Total Omega-3 FA Cmax of at least 110% of the EPA+DHA Cmax or Total Omega-3 FA Cmax for Epanova under the same administration conditions
153. A method of administration or treatment under fasting conditions according to one of the preferred embodiments 47 through 49 resulting in an EPA+DHA Cmax or Total Omega-3 FA Cmax of at least 120% of the EPA+DHA Cmax or Total Omega-3 FA Cmax for Epanova under the same administration conditions
154. A method of administration or treatment under fasting conditions according to one of the preferred embodiments 47 through 49 resulting in an EPA+DHA Cmax or Total Omega-3 FA Cmax of at least 130% of the EPA+DHA Cmax or Total Omega-3 FA Cmax for Epanova under the same administration conditions
155. A method of administration or treatment under fasting conditions according to one of the preferred embodiments 47 through 49 resulting in an EPA+DHA Cmax or Total Omega-3 FA Cmax of at least 140% of the EPA+DHA Cmax or Total Omega-3 FA Cmax for Epanova under the same administration conditions
156. A method of administration or treatment under fasting conditions according to one of the preferred embodiments 47 through 49 resulting in an EPA+DHA Cmax or Total Omega-3 FA Cmax of at least 150% of the EPA+DHA Cmax or Total Omega-3 FA Cmax for Epanova under the same administration conditions
157. A method of administration or treatment under high fat meal conditions according to one of the preferred embodiments 47 through 49 resulting in an EPA+DHA AUC or Total Omega-3 FA AUC of at least 100% of the EPA+DHA AUC or Total Omega-3 FA AUC for Epanova under the same administration conditions
158. A method of administration or treatment under high fat meal conditions according to one of the preferred embodiments 47 through 49 resulting in an EPA+DHA AUC or Total Omega-3 FA AUC of at least 105% of the EPA+DHA AUC or Total Omega-3 FA AUC for Epanova under the same administration conditions
159. A method of administration or treatment under high fat meal conditions according to one of the preferred embodiments 47 through 49 resulting in an EPA+DHA AUC or Total Omega-3 FA AUC of at least 110% of the EPA+DHA AUC or Total Omega-3 FA AUC for Epanova under the same administration conditions
160. A method of administration or treatment under high fat meal conditions according to one of the preferred embodiments 47 through 49 resulting in an EPA+DHA AUC or Total Omega-3 FA AUC of at least 120% of the EPA+DHA AUC or Total Omega-3 FA AUC for Epanova under the same administration conditions
161. A method of administration or treatment under high fat meal conditions according to one of the preferred embodiments 47 through 49 resulting in an EPA+DHA AUC or Total Omega-3 FA AUC of at least 130% of the EPA+DHA AUC or Total Omega-3 FA AUC for Epanova under the same administration conditions
162. A method of administration or treatment under high fat meal conditions according to one of the preferred embodiments 47 through 49 resulting in an EPA+DHA AUC or Total Omega-3 FA AUC of at least 140% of the EPA+DHA AUC or Total Omega-3 FA AUC for Epanova under the same administration conditions
163. A method of administration or treatment under low fat meal conditions according to one of the preferred embodiments 47 through 49 resulting in an EPA+DHA AUC or Total Omega-3 FA AUC of at least 100% of the EPA+DHA AUC or Total Omega-3 FA AUC for Epanova under the same administration conditions
164. A method of administration or treatment under low fat meal conditions according to one of the preferred embodiments 47 through 49 resulting in an EPA+DHA AUC or Total Omega-3 FA AUC of at least 105% of the EPA+DHA AUC or Total Omega-3 FA AUC for Epanova under the same administration conditions
165. A method of administration or treatment under low fat meal conditions according to one of the preferred embodiments 47 through 49 resulting in an EPA+DHA AUC or Total Omega-3 FA AUC of at least 110% of the EPA+DHA AUC or Total Omega-3 FA AUC for Epanova under the same administration conditions
166. A method of administration or treatment under low fat meal conditions according to one of the preferred embodiments 47 through 49 resulting in an EPA+DHA AUC or Total Omega-3 FA AUC of at least 120% of the EPA+DHA AUC or Total Omega-3 FA AUC for Epanova under the same administration conditions
167. A method of administration or treatment under low fat meal conditions according to one of the preferred embodiments 47 through 49 resulting in an EPA+DHA AUC or Total Omega-3 FA AUC of at least 130% of the EPA+DHA AUC or Total Omega-3 FA AUC for Epanova under the same administration conditions
168. A method of administration or treatment under low fat meal conditions according to one of the preferred embodiments 47 through 49 resulting in an EPA+DHA AUC or Total Omega-3 FA AUC of at least 140% of the EPA+DHA AUC or Total Omega-3 FA AUC for Epanova under the same administration conditions
169. A method of administration or treatment under fasting conditions according to one of the preferred embodiments 47 through 49 resulting in an EPA+DHA AUC or Total Omega-3 FA AUC of at least 100% of the EPA+DHA AUC or Total Omega-3 FA AUC for Epanova under the same administration conditions
170. A method of administration or treatment under fasting conditions according to one of the preferred embodiments 47 through 49 resulting in an EPA+DHA AUC or Total Omega-3 FA AUC of at least 105% of the EPA+DHA AUC or Total Omega-3 FA AUC for Epanova under the same administration conditions
171. A method of administration or treatment under fasting conditions according to one of the preferred embodiments 47 through 49 resulting in an EPA+DHA AUC or Total Omega-3 FA AUC of at least 110% of the EPA+DHA AUC or Total Omega-3 FA AUC for Epanova under the same administration conditions
172. A method of administration or treatment under fasting conditions according to one of the preferred embodiments 47 through 49 resulting in an EPA+DHA AUC or Total Omega-3 FA AUC of at least 120% of the EPA+DHA AUC or Total Omega-3 FA AUC for Epanova under the same administration conditions
173. A method of administration or treatment under fasting conditions according to one of the preferred embodiments 47 through 49 resulting in an EPA+DHA AUC or Total Omega-3 FA AUC of at least 130% of the EPA+DHA AUC or Total Omega-3 FA AUC for Epanova under the same administration conditions
174. A method of administration or treatment under fasting conditions according to one of the preferred embodiments 47 through 49 resulting in an EPA+DHA AUC or Total Omega-3 FA AUC of at least 140% of the EPA+DHA AUC or Total Omega-3 FA AUC for Epanova under the same administration conditions
175. A method of administration or treatment under fasting conditions according to one of the preferred embodiments 47 through 49 resulting in an EPA+DHA AUC or Total Omega-3 FA AUC of at least 150% of the EPA+DHA AUC or Total Omega-3 FA AUC for Epanova under the same administration conditions
176. A method of treatment according to one of the preferred embodiments 47 through 49, in which the subject is a patient diagnosed with very high triglycerides (equal or more than 500 mg/dL).
177. A method of treatment according to one of the preferred embodiments 47 through 49, in which the subject is a patient diagnosed with high triglycerides (equal or more than 200 mg/dL but less than 500 mg/dL).
178. A method of treatment according to one of the preferred embodiments 47 through 49, in which the subject is a patient already undergoing treatment with a statin and then diagnosed with high triglycerides (equal or more than 200 mg/dL but less than 500 mg/dL).
179. A method of treatment according to one of the preferred embodiments 47 through 49, in which the subject is a patient diagnosed with mixed dyslipidemia (TG equal or more than 200 mg/dL and LDL-cholesterol equal or more than 190 mg/dL).
180. A method of treatment according to one of the preferred embodiments 47 through 49, in which the subject is a patient diagnosed/assessed to be at substantially elevated risk for cardiovascular events.
181. A method of treatment according to one of the preferred embodiments 47 through 49, in which the subject is a patient diagnosed with diabetes.
182. A method of treatment according to one of the preferred embodiments 47 through 49, in which the subject is a patient diagnosed with pre-diabetes or metabolic syndrome.
183. A method of treatment according to one of the preferred embodiments 176 through 182, in which the treatment results in significant reduction of blood, serum or plasma triglyceride levels.
184. A method of treatment according to one of the preferred embodiments 176 through 182, in which the treatment results in significant reduction of blood, serum or plasma triglyceride levels while not significantly increasing blood, serum or plasma LDL-cholesterol levels.
185. A method of treatment according to one of the preferred embodiments 176 through 182, in which the treatment results in significant reduction of blood, serum or plasma total-cholesterol levels.
186. A method of treatment according to one of the preferred embodiments 176 through 182, in which the treatment results in significant reduction of blood, serum or plasma non-HDL-cholesterol levels.
187. A method of treatment according to one of the preferred embodiments 176 through 182, in which the treatment results in significant reduction of blood, serum or plasma LDL-cholesterol levels.
188. A method of treatment according to one of the preferred embodiments 176 through 182, in which the treatment results in significant reduction of blood, serum or plasma VLDL-cholesterol levels.
189. A method of treatment according to one of the preferred embodiments 176 through 182, in which the treatment results in significant reduction of blood, serum or plasma VLDL-cholesterol levels while not significantly increasing blood, serum or plasma LDL-cholesterol levels.
190. A method of treatment according to one of the preferred embodiments 176 through 182, in which the treatment results in significant reduction of blood, serum or plasma apo-B levels.
191. A method of treatment according to one of the preferred embodiments 176 through 182, in which the treatment results in significant reduction of blood, serum or plasma LP-PLA2 levels.
192. A method of treatment according to one of the preferred embodiments 176 through 182, in which the treatment results in significant reduction of blood, serum or plasma hs-CRP levels.
193. A method of treatment according to one of the preferred embodiments 176 through 182, in which the treatment results in significant increase of blood, serum or plasma HDL-cholesterol levels.
194. A method of treatment according to one of the preferred embodiments 176 through 182, in which the treatment results in significant increase of blood, serum or plasma apo-A levels.
195. A method of treatment according to one of the preferred embodiments 176 through 182, in which the treatment results in significant reduction of the risk of suffering certain cardiovascular events.

What is claimed:

1. An orally administrable pharmaceutical composition comprising at least 30% omega-3 fatty acids and one or more additional components selected from the group consisting of absorption enhancers and adjuvants,
wherein the absorption enhancer is selected from the group consisting of: phenolic antioxidants, hexadecyldimethylbenzaylammonium chloride, and hexasalicylic acid, and
wherein the adjuvant is selected from the group consisting of surfactants and emulsifiers.

2. The orally administrable pharmaceutical composition of claim 1, wherein the phenolic antioxidant is selected from the group consisting of: propyl gallate, butylated hydroxyansole (BHA), butylated hydroxytoluene (BHT), tert-butyl-hydroquinone (TBHQ), 4-hydroxymethyl-2,6-di-ter-butylphenol (HMBP), 2,4,5-trihydroxybutyrophenone (THBP), gallic acid, alpha-tocopherol and esters, derivatives, analogues or equivalents thereof.

3. The orally administrable pharmaceutical composition of claim 1, wherein the pharmaceutical composition comprises an absorption enhancer selected from the group consisting of: propyl gallate, butylated hydroxyanisole (BHA), butylated hydroxytoluene (BHT), tertiary butylhydroquinone (TBHQ), 2,4,5-trihydroxybutyrophenone (THBP), alpha-tocopheryl polyethylene glycol succinate, hexadecyldimethylbenzylammonium chloride, and hexylsalicylic acid.

4. The orally administrable pharmaceutical composition of claim 1, wherein the adjuvant is selected from the group consisting of: non-ionic surfactants and oleic acid and salts or esters thereof.

5. The orally administrable pharmaceutical composition of claim 1, wherein the adjuvant is selected from the group consisting of: glyceryl oleate, sorbitan oleate, polyglyceryl-3-oleate, sodium oleate, and ethyl oleate.

6. The orally administrable pharmaceutical composition of claim 1, wherein the pharmaceutical composition comprises one or more absorption enhancers.

7. The orally administrable pharmaceutical composition of claim 1, wherein the pharmaceutical composition comprises one or more absorption enhancers and one or more adjuvants.

8. The orally administrable pharmaceutical composition of claim 1, wherein the pharmaceutical composition comprises a hard or soft gelatin capsule.

9. The orally administrable pharmaceutical composition of claim 1, wherein the hard or soft gelatin capsule comprises an oil comprising one or more omega-3 fatty acids.

10. The orally administrable pharmaceutical composition of claim 9, wherein the oil comprises one or more absorption enhancers.

11. The orally administrable pharmaceutical composition of claim 8, wherein the hard or soft gelatin capsule comprises a capsule shell containing one or more absorption enhancers.

12. The orally administrable pharmaceutical composition of claim 1, wherein the pharmaceutical composition comprises at least one of the following absorption enhancers:
a. propyl gallate, in an amount of up to 5 mg/g,
b. butylated hydroxyanisole (BHA) in an amount of up to 5 mg/g,
c. butylated hydroxytoluene (BHT) in an amount of up to 5 mg/g,
d. tertiary butylhydroquinone (TBHQ) in an amount of up to 5 mg/g,
e. 2,4,5-trihydroxybutyrophenone (THBP) in an amount of up to 5 mg/g,
f. alpha-tocopheryl polyethylene glycol succinate (TPGS, tocophersolan) in an amount of up to 20 mg/g,
g. hexadecyldimethylbenzylammonium chloride (BAC) in an amount of up to 20 mg/g, and
h. hexylsalicylic acid (HSA) in an amount of up to 20 mg/g.

13. The orally administrable pharmaceutical composition of claim 1, wherein the pharmaceutical composition comprises at least 65% omega-3 fatty acids.

14. The orally administrable pharmaceutical composition of claim 1, wherein the pharmaceutical composition comprises at least 80% omega-3 fatty acids.

15. The orally administrable pharmaceutical composition of claim 1, wherein the pharmaceutical composition comprises at least 85% omega-3 fatty acids.

16. The orally administrable pharmaceutical composition of claim 1, wherein the omega-3 fatty acids comprises eicosapentaenoic acid (EPA; C20:5-n3) and docosapentaenoic acid (DPA; C22:5-n3), wherein the ratio of DHA to EPA (DHA:EPA) is less than 1:10, and wherein the ratio of DHA to DPA (DHA:DPA) is less than 2:1.

17. The orally administrable pharmaceutical composition of claim 1, wherein the pharmaceutical composition comprises eicosapentaenoic acid (EPA) in an amount between about 70% to about 95% of the total amount of fatty acids and docosapentaenoic acid (DPA), wherein the composition comprises no more than 5% docosahexaenoic acid (DHA) of the total amount of fatty acids, and wherein the ratio of DHA:DPA is 1:1 or lower.

18. The orally administrable pharmaceutical composition of claim 1, wherein the pharmaceutical composition comprises eicosapentaenoic acid (EPA) and docosapentaenoic acid (DPA), wherein the amount of EPA and DPA is about 55% or more by weight, and wherein the ratio of docosahexaenoic acid (DHA) to DPA (DHA:DPA) is no more than 1.1.

19. The orally administrable pharmaceutical composition of claim 1, wherein the pharmaceutical composition comprises docosapentaenoic acid (DPA) and docosahexaenoic acid (DHA) in an amount of at least 60% of the total amount of the fatty acids present in the composition, wherein the ratio of DPA to DHA (DPA:DHA) is between about 10:1 to about 1:10.

20. A method of treating a condition in a subject in need thereof, comprising administering to the subject the orally administrable pharmaceutical composition of claim 1,
wherein the condition is selected from the group consisting of: hypertriglyceridemia; hypercholesterolemia; mixed dyslipidemia; coronary heart disease (CHD); vascular disease; cardiovascular disease; acute coronary syndrome; atherosclerotic disease and related conditions; heart failure; cardiac arrhythmias; coagulatory conditions associated with cardiac arrhythmias; ischemic dementia; vascular dementia; hypertension; coagulation related disorders; nephropathy; kidney or urinary tract disease; retinopathy; cognitive and other CNS disorders; autoimmune diseases; inflammatory diseases; asthma or other respiratory disease; dermatological disease; metabolic syndrome; diabetes, diabetes mellitis or other form of metabolic disease; liver disease; non-alcoholic fatty liver disease; disease of the gastrointestinal tract; disease of the male or female reproductive system or related secondary sexual organs; a cancer of any type, including lymphomas and myelomas; and an infection caused by a virus, bacterium, fungus, protozoa or other organism.

21. An orally administrable composition comprise docosapentaenoic acid (DPA) and docosahexaenoic acid (DHA) in an amount of at least 60% of the total amount of the fatty acids present in the composition, wherein the ratio of DPA to DHA (DPA:DHA) is between about 10:1 to about 1:10, and
wherein the composition further comprises one or more additional components selected from the group consisting of absorption enhancers and adjuvants,
wherein the absorption enhancer is selected from the group consisting of: phenolic antioxidants, hexadecyldimethylbenzaylammonium chloride, and hexasalicylic acid, and
wherein the adjuvant is selected from the group consisting of surfactants and emulsifiers.

22. The orally administrable pharmaceutical composition of claim 21, wherein the phenolic antioxidant is selected from the group consisting of: propyl gallate, butylated hydroxyansole (BHA), butylated hydroxytoluene (BHT), tert-butyl-hydroquinone (TBHQ), 4-hydroxymethyl-2,6-di-ter-butylphenol (HMBP), 2,4,5-trihydroxybutyrophenone (THBP), gallic acid, alpha-tocopherol and esters, derivatives, analogues or equivalents thereof.

23. The orally administrable pharmaceutical composition of claim 21, wherein the pharmaceutical composition comprises an absorption enhancer selected from the group consisting of: propyl gallate, butylated hydroxyanisole (BHA), butylated hydroxytoluene (BHT), tertiary butylhydroquinone (TBHQ), 2,4,5-trihydroxybutyrophenone (THBP), alpha-tocopheryl polyethylene glycol succinate, hexadecyldimethylbenzylammonium chloride, and hexylsalicylic acid.

24. The orally administrable pharmaceutical composition of claim 21, wherein the adjuvant is selected from the group consisting of: non-ionic surfactants and oleic acid and salts or esters thereof.

25. The orally administrable pharmaceutical composition of claim 21, wherein the adjuvant is selected from the group consisting of: glyceryl oleate, sorbitan oleate, polyglyceryl-3-oleate, sodium oleate, and ethyl oleate.

26. The orally administrable pharmaceutical composition of claim 21, wherein the pharmaceutical composition comprises one or more absorption enhancers.

27. The orally administrable pharmaceutical composition of claim 21, wherein the pharmaceutical composition comprises one or more absorption enhancers and one or more adjuvants.

28. The orally administrable pharmaceutical composition of claim 21, wherein the pharmaceutical composition comprises a hard or soft gelatin capsule.

29. The orally administrable pharmaceutical composition of claim 21, wherein the hard or soft gelatin capsule comprises an oil comprising one or more omega-3 fatty acids.

30. The orally administrable pharmaceutical composition of claim 29, wherein the oil comprises one or more absorption enhancers.

31. The orally administrable pharmaceutical composition of claim 28, wherein the hard or soft gelatin capsule comprises a capsule shell containing one or more absorption enhancers.

32. The orally administrable pharmaceutical composition of claim 21, wherein the pharmaceutical composition comprises at least one of the following absorption enhancers:
i. propyl gallate, in an amount of up to 5 mg/g,
j. butylated hydroxyanisole (BHA) in an amount of up to 5 mg/g,
k. butylated hydroxytoluene (BHT) in an amount of up to 5 mg/g,
l. tertiary butylhydroquinone (TBHQ) in an amount of up to 5 mg/g,
m. 2,4,5-trihydroxybutyrophenone (THBP) in an amount of up to 5 mg/g,
n. alpha-tocopheryl polyethylene glycol succinate (TPGS, tocophersolan) in an amount of up to 20 mg/g,
o. hexadecyldimethylbenzylammonium chloride (BAC) in an amount of up to 20 mg/g, and
p. hexylsalicylic acid (HSA) in an amount of up to 20 mg/g.

33. The orally administrable pharmaceutical composition of claim 21, wherein the composition comprises DPA and DHA in an amount of at least 75% of the total amount of the fatty acids present in the composition.

34. The orally administrable pharmaceutical composition of claim 21, wherein the composition comprises DPA and DHA in an amount of at least 80% of the total amount of the fatty acids present in the composition.

35. The orally administrable pharmaceutical composition of claim 21, wherein the ratio of DPA to DHA (DPA:DHA) is between about 5:1 to about 1:5.

36. The orally administrable pharmaceutical composition of claim 21, wherein the ratio of DPA to DHA (DPA:DHA) is between about 2:1 to about 10:1.

37. The orally administrable pharmaceutical composition of claim 21, wherein the ratio of DPA to DHA (DPA:DHA) is between about 2:1 to about 8:1.

38. The orally administrable pharmaceutical composition of claim 21, wherein the ratio of DPA to DHA (DPA:DHA) is between about 2:1 to about 5:1.

39. The orally administrable pharmaceutical composition of claim 21, wherein the ratio of DPA to DHA (DPA:DHA) is between about 3:1 to about 5:1.

40. The orally administrable pharmaceutical composition of claim 21, wherein the ratio of DHA to DPA (DHA:DPA) is between about 2:1 to about 10:1.

41. The orally administrable pharmaceutical composition of claim 21, wherein the ratio of DHA to DPA (DHA:DPA) is between about 2:1 to about 8:1.

42. The orally administrable pharmaceutical composition of claim 21, wherein the ratio of DHA to DPA (DHA:DPA) is between about 2:1 to about 5:1.

43. The orally administrable pharmaceutical composition of claim 21, wherein the ratio of DHA to DPA (DHA:DPA) is between about 3:1 to about 5:1.

44. A method of treating a condition in a subject in need thereof, comprising administering to the subject the orally administrable pharmaceutical composition of claim 21,
wherein the condition is selected from the group consisting of: hypertriglyceridemia; hypercholesterolemia; mixed dyslipidemia; coronary heart disease (CHD); vascular disease; cardiovascular disease; acute coronary syndrome; atherosclerotic disease and related conditions; heart failure; cardiac arrhythmias; coagulatory conditions associated with cardiac arrhythmias; ischemic dementia; vascular dementia; hypertension; coagulation related disorders; nephropathy; kidney or urinary tract disease; retinopathy; cognitive and other CNS disorders; autoimmune diseases; inflammatory diseases; asthma or other respiratory disease; dermatological disease; metabolic syndrome; diabetes, diabetes mellitis or other form of metabolic disease; liver disease; non-alcoholic fatty liver disease; disease of the gastrointestinal tract; disease of the male or female reproductive system or related secondary sexual organs; a cancer of any type, including lymphomas and myelomas; and an infection caused by a virus, bacterium, fungus, protozoa or other organism.
